(19) United States
(12) Patent Application Publication
(10) Pub. No.: US 2011/0172193 A1
(43) Pub. Date: Jul. 14, 2011

(54) PRO-DRUGS OF (E)-7-(3-(2-AMINO-1-FLUOROETHYLIDENE)PIPERIDIN-1-YL)-1-CYCLOPROPYL-6-FLUORO-8-METHOXY-4-OXO-1,4-DIHYDROQUINOLINE-3-CARBOXYLIC ACID

(76) Inventors: Mark MACIELAG, Branchburg, NJ (US); Gary Eichenbaum, Belle Mead, NJ (US); Tim Gaekens, Wilrijk (BE); Michel Guillaume, Steenokkerzeel (BE); Brian Shook, Gilbertsville, PA (US)

(21) Appl. No.: 12/983,365

(22) Filed: Jan. 3, 2011

Related U.S. Application Data
(60) Provisional application No. 61/293,870, filed on Jan. 11, 2010.

(51) Int. Cl. A61K 31/675; C07D 401/02; A61K 31/4709; C07D 401/14; C07D 405/14; C07F 9/06; A61P 31/04

(52) U.S. Cl. 514/82; 546/156; 514/312; 546/23

(57) ABSTRACT

The present invention is directed to pro-drugs of (E)-7-(3-(2-amino-1-fluoroethylidene)piperidin-1-yl)-1-cyclopropyl-6-fluoro-8-methoxy-4-oxo-1,4-dihydroquinoline-3-carboxylic acid, pharmaceutical compositions containing them and the use of said pro-drugs and pharmaceutical compositions as antimicrobial agents against pathogenic microorganisms, particularly against resistant microbes.

### CROSS-REFERENCE TO RELATED APPLICATIONS

The present application claims the benefits of the filing of U.S. Provisional Application No. 61/293,870 filed Jan. 11, 2010. The complete disclosures of the aforementioned related patent applications are hereby incorporated herein by reference for all purposes.

### FIELD OF THE INVENTION

The present invention is directed to pro-drugs of (E)-7-(3-(2-amino-1-fluoroethylidene)piperidin-1-yl)-1-cyclopropyl-6-fluoro-8-methoxy-4-oxo-1,4-dihydroquinoline-3-carboxylic acid, pharmaceutical compositions containing them and the use of said pro-drugs and pharmaceutical compositions as antimicrobial agents against pathogenic microorganisms, particularly against resistant microbes.

### BACKGROUND OF THE INVENTION

Grant III, E. B., et al., in U.S. Pat. No. 7,179,805 B2, issued Feb. 20, 2007 discloses 7-amino alkylidenyl-heterocyclic quinolones and naphthyridones useful as antimicrobial agents.

### SUMMARY OF THE INVENTION

The present invention is directed to compounds of formula (I)

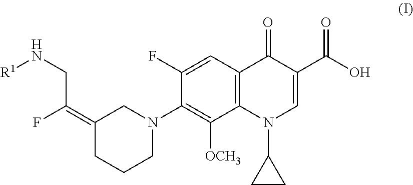

wherein

R¹ is selected from the group consisting of

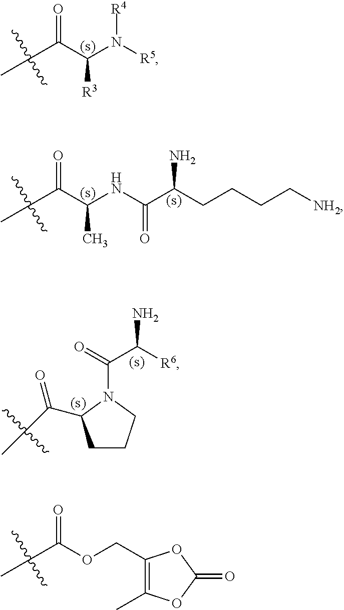

### and —P(O)(OR⁷)₂;

R³ is selected from the group consisting of hydrogen, lower alkyl, benzyl, —CH₂CO₂H, —(CH₂)₄NH₂ and —CH₂N(CH₃)₂;

R⁴ is selected from the group consisting of hydrogen and lower alkyl;

R⁵ is selected from the group consisting of hydrogen,

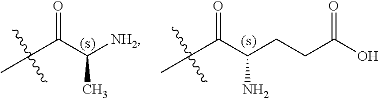

### and —C(O)—(CH₂)₂—C(O)-mPEG(2000);

R⁶ is selected from the group consisting of lower alkyl and —(CH₂)₄—NH₂;

each R⁷ is independently selected from lower alkyl;

and pharmaceutically acceptable salts thereof.

The present invention is further directed to compounds of formula (II)

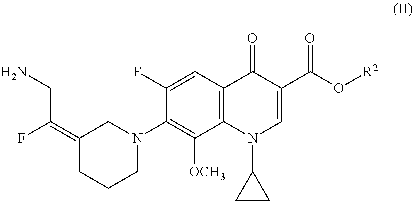

wherein R² is lower alkyl; and pharmaceutically acceptable salts thereof.

The present invention is further directed to processes for the preparation of the compounds of formula (I). The present invention is further directed to processes for the preparation of the compounds of formula (II). The present invention is further directed to a product prepared according to the process described herein.

Illustrative of the invention is a pharmaceutical composition comprising a pharmaceutically acceptable carrier and a compound of formula (I) or a compound of formula (II) as described herein. An illustration of the invention is a pharmaceutical composition made by mixing a compound of formula (I) or a compound of formula (II) as described herein and a pharmaceutically acceptable carrier. Illustrating the invention is a process for making a pharmaceutical composition comprising mixing a compound of formula (I) or a compound of formula (II) as described herein and a pharmaceutically acceptable carrier.

It has been found that the compounds of the present invention and compositions containing said compounds, are effective antimicrobial agents when administered to mammals by virtue of their conversion to an agent with activity against a broad range of pathogenic microorganisms with advantages of activity against resistant microbes. Moreover, it has been found that the compounds of the present invention have suitable pharmaceutical properties, including adequate aqueous solubility for intravenous administration at a pharmaceutically acceptable pH. Accordingly, the present invention is further directed to a method of treating a subject having a condition caused by or contributed to by bacterial infection, comprising administering to said mammal a therapeutically effective amount of a compound of formula (I) or a compound of formula (II) as described herein.

The present invention is further directed to a method of preventing a subject from suffering from a condition caused by or contributed to by bacterial infection, which comprises administering to the subject a prophylactically effective dose of the pharmaceutical composition of a compound of formula (I) or a compound of formula (II).

The present invention is further directed to the use of a compound of formula (I) or a compound of formula (II) for the preparation of a medicament for treating and/or preventing a condition caused by or contributed to by bacterial infection, in a subject in need thereof. In an embodiment, the present invention is directed to the use of a compound of formula (I) or a compound of formula (II) for the preparation of a medicament for treating and/or preventing a condition caused by or contributed to by bacterial infection associated with a drug resistant bacteria, in a subject in need thereof.

### DETAILED DESCRIPTION OF THE INVENTION

The present invention is directed to compounds of formula (I)

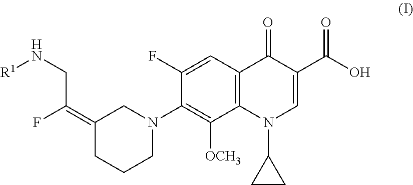

wherein R¹ is as herein defined, and pharmaceutically acceptable salts thereof.

The present invention is further directed to compounds of formula (II)

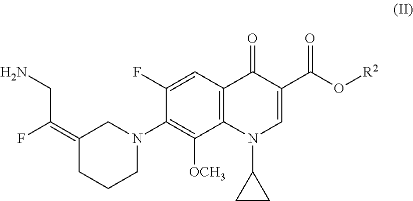

wherein R² is as herein defined, and pharmaceutically acceptable salts thereof. The compounds of formula (I) and the compounds of formula (II) are pro-drugs, which when administered to a mammal, convert to the compound of formula (M)

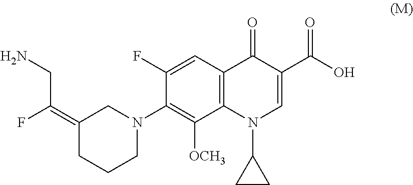

also known as (E)-7-(3-(2-amino-1-fluoroethylidene)piperidin-1-yl)-1-cyclopropyl-6-fluoro-8-methoxy-4-oxo-1,4-dihydroquinoline-3-carboxylic acid. Thus the compounds of formula (I) and compounds of formula (II) are useful for treatment of infections or infectious diseases caused by pathogenic microorganisms, preferably, resistant microbes.

In an embodiment of the present invention, R¹ is

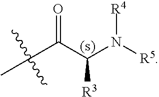

In another embodiment of the present invention, R¹ is

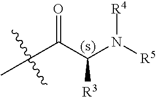

and the stereo-center is preferably present in an enantiomeric excess of the (S) stereo-configuration of greater than or equal to about 75% ee, preferably, in an enantiomeric excess of the (S) stereo-configuration greater than or equal to about 85% ee, more preferably, in an enantiomeric excess of the (S) stereo-configuration greater than or equal to about 95% ee, more preferably still, in an enantiomeric excess of the (S) stereo-configuration greater than or equal to about 99% ee.

In an embodiment of the present invention, R³ is selected from the group consisting of hydrogen, methyl, isopropyl, isobutyl, benzyl, —CH₂CO₂H, —(CH₂)₄NH₂ and —CH₂N(CH₃)₂.

In an embodiment of the present invention R⁴ is selected from the group consisting of hydrogen, methyl and ethyl. In another embodiment of the present invention, R⁴ is selected from the group consisting of hydrogen and methyl. In another embodiment of the present invention, R⁴ is hydrogen.

In an embodiment of the present invention, R⁵ is selected from the group consisting of hydrogen,

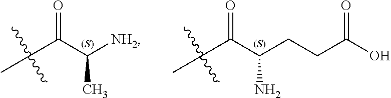

and —C(O)—(CH₂)₂—C(O)-mPEG(2000). In another embodiment of the present invention, R⁵ is selected from the group consisting of hydrogen,

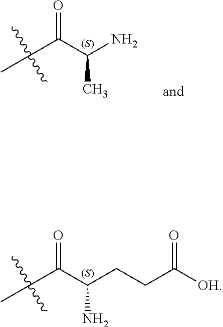

In an embodiment of the present invention, R⁵ is

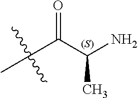

and the stereo-center is present in an enantiomeric excess of the (S) stereo-configuration of greater than or equal to about 75% ee, preferably, in an enantiomeric excess of the (S) stereo-configuration greater than or equal to about 85% ee, more preferably, in an enantiomeric excess of the (S) stereo-configuration greater than or equal to about 95% ee, more preferably still, in an enantiomeric excess of the (S) stereo-configuration greater than or equal to about 99% ee.

In another embodiment of the present invention, R⁵ is

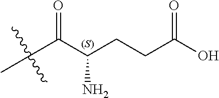

and the stereo-center is present in an enantiomeric excess of the (S) stereo-configuration of greater than or equal to about 75% ee, preferably, in an enantiomeric excess of the (S) stereo-configuration greater than or equal to about 85% ee, more preferably, in an enantiomeric excess of the (S) stereo-configuration greater than or equal to about 95% ee, more preferably still, in an enantiomeric excess of the (S) stereo-configuration greater than or equal to about 99% ee.

In an embodiment of the present invention, R¹ is

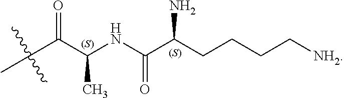

In an embodiment of the present invention, R¹ is

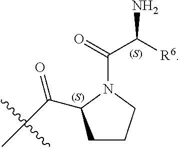

In an embodiment of the present invention, R⁶ is selected from the group consisting of methyl, isopropyl and —(CH₂)₄NH₂. In another embodiment of the present invention, R¹ is

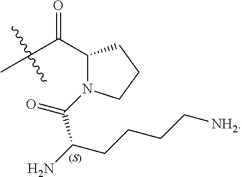

In an embodiment of the present invention, R¹ is

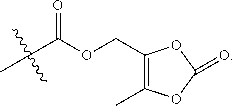

In another embodiment of the present invention, R¹ is

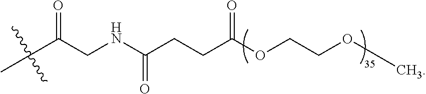

In an embodiment of the present invention, R¹ is selected from the group consisting of

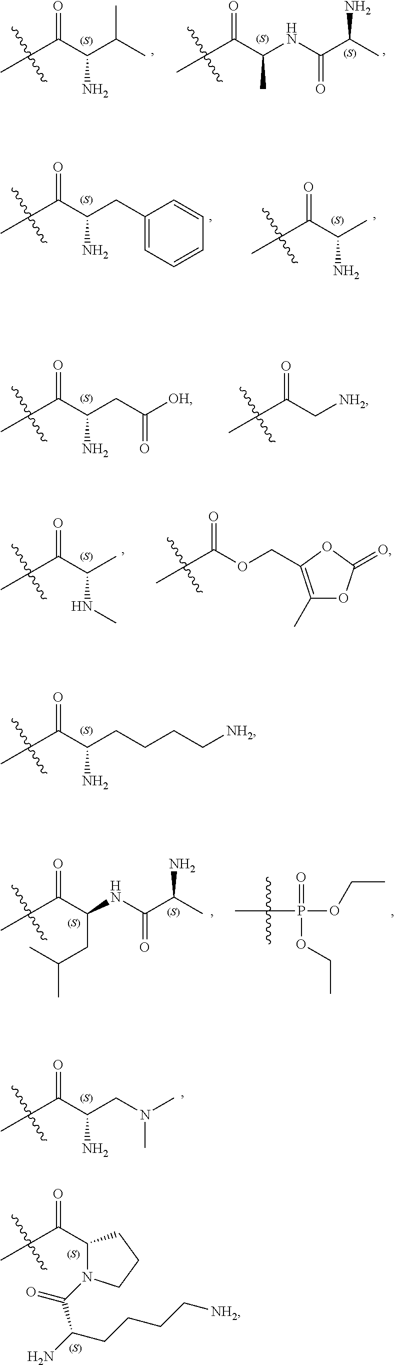

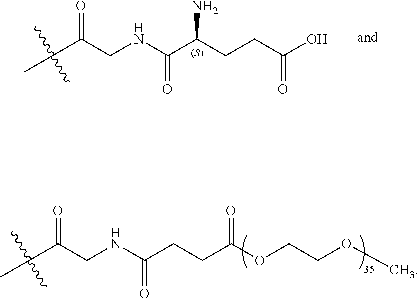

In another embodiment of the present invention, R¹ is selected from the group consisting of

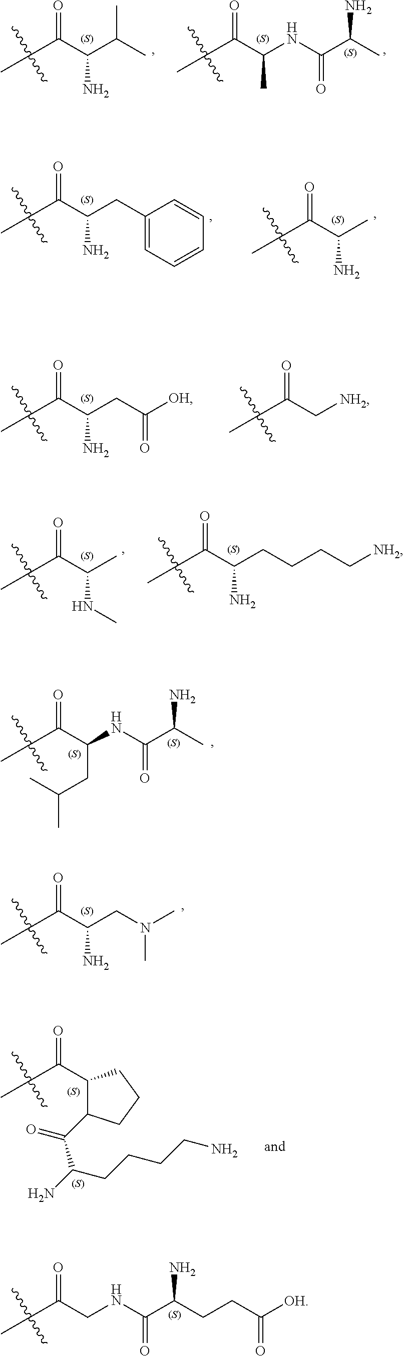

In another embodiment of the present invention, R¹ is selected from the group consisting of

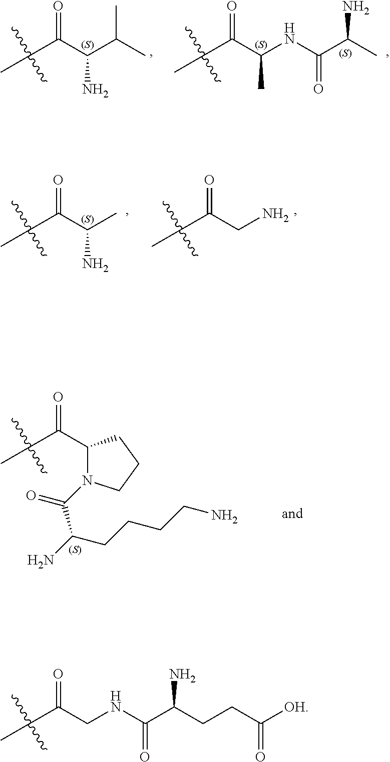

In another embodiment of the present invention, R¹ is selected from the group consisting of

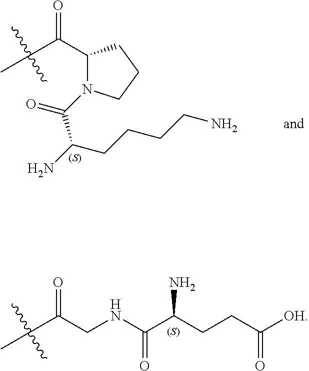

In an embodiment of the present invention, R¹ is selected from the group consisting of —PO(OR⁷)₂ wherein each R⁷ is independently selected from lower alkyl. In another embodiment of the present invention, each R⁷ is selected from the group consisting of methyl, ethyl, isopropyl and t-butyl. In another embodiment of the present invention, both R⁷ groups are the same and are selected from lower alkyl. In another embodiment of the present invention, both R⁷ groups are the same and are selected from the group consisting of methyl, ethyl, isopropyl and t-butyl. In another embodiment of the present invention, both R⁷ groups are the same and are ethyl.

In an embodiment of the present invention, R² is selected from the group consisting of methyl, ethyl, isopropyl, isobutyl and t-butyl. In another embodiment of the present invention, R² is selected from the group consisting of ethyl and isopropyl.

Additional embodiments of the present invention, include those wherein the substituents selected for one or more of the variables defined herein (i.e. R¹, R², R³, R⁴, R⁵, R⁶ and R⁷) are independently selected to be any individual substituent or any subset of substituents selected from the complete list as defined herein. Representative compounds of the present invention are as listed in Table 1 to 2, below. In another embodiment of the present invention is any single compound or subset of compounds selected from the representative compounds listed in Tables 1-2, below.

TABLE 1
Representative Compounds of Formula (I)

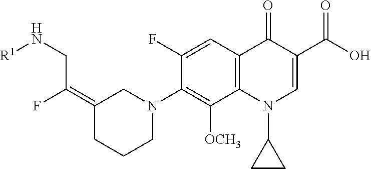

| ID No. | R¹ | (Type) |
|---|---|---|
| 1 | 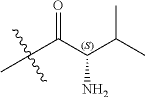 | L-Val |
| 2 | 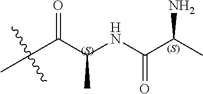 | L-Ala-L-Ala |
| 3 | 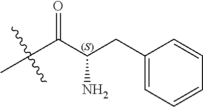 | L-Phe |
| 4 | 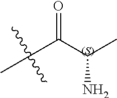 | L-Ala |
| 5 | 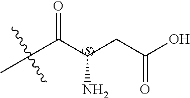 | L-Asp |
| 6 | 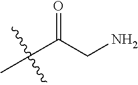 | Gly |
| 7 | 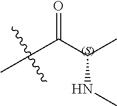 | N-Me-L-Ala |
| 8 | 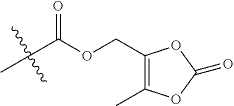 | 5-Methyl-2- oxo-1,3-dioxol- 4-ylmethyl- carbamoyl |
| 9 | 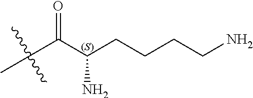 | L-Lys |
| 10 | 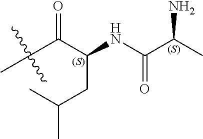 | L-Ala-L-Leu |
| 13 | 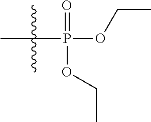 | Diethyl- phosphoryl |
| 14 | 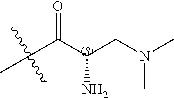 | L-Azaleucine |
| 15 | 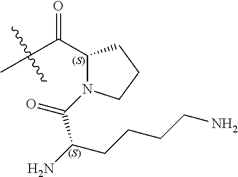 | L-Lys-L-Pro |
| 16 | 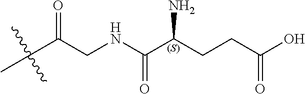 | L-Glu-Gly |
| 17 | 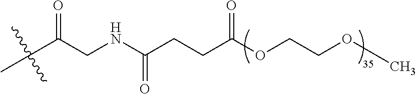 | mPEG(2000)- succinyl-Gly |

TABLE 2
Representative Compounds of Formula (II)

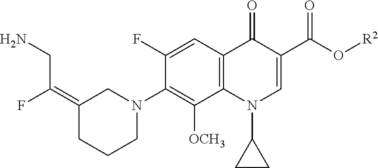

| ID No. | R² | (Type) |
|---|---|---|
| 11 | ethyl | ethyl ester |
| 12 | isopropyl | isopropyl ester |

In additional embodiments, the present invention is directed to one or more compounds of formula (I) selected from the group consisting of
7-{3-[2-((2S)-2-Amino-3-methyl-butyrylamino)-1-fluoro-ethylidene]-piperidin-1-yl}-1-cyclopropyl-6-fluoro-8-methoxy-4-oxo-1,4-dihydro-quinoline-3-carboxylic acid hydrochloride (Compound 1);
7-(3-{2-[(2S)-2-((2S)-2-Amino-propionylamino)-propionylamino]-1-fluoro-ethylidene}-piperidin-1-yl)-1-cyclopropyl-6-fluoro-8-methoxy-4-oxo-1,4-dihydro-quinoline-3-carboxylic acid hydrochloride (Compound 2);
7-{3-[2-((2S)-2-Amino-3-phenyl-propionylamino)-1-fluoro-ethylidene]-piperidin-1-yl}-1-cyclopropyl-6-fluoro-8-methoxy-4-oxo-1,4-dihydro-quinoline-3-carboxylic acid hydrochloride (Compound 3);
7-{3-[2-((2S)-2-Amino-propionylamino)-1-fluoro-ethylidene]-piperidin-1-yl}-1-cyclopropyl-6-fluoro-8-methoxy-4-oxo-1,4-dihydro-quinoline-3-carboxylic acid hydrochloride (Compound 4);
7-{3-[2-((2S)-2-Amino-3-carboxy-propionylamino)-1-fluoro-ethylidene]-piperidin-1-yl}-1-cyclopropyl-6-fluoro-8-methoxy-4-oxo-1,4-dihydro-quinoline-3-carboxylic acid hydrochloride (Compound 5);
7-{3-[2-(2-Amino-acetylamino)-1-fluoro-ethylidene]-piperidin-1-yl}-1-cyclopropyl-6-fluoro-8-methoxy-4-oxo-1,4-dihydro-quinoline-3-carboxylic acid hydrochloride (Compound 6);
1-Cyclopropyl-6-fluoro-7-{3-[1-fluoro-2-((2S)-2-methylamino-propionylamino)-ethylidene]-piperidin-1-yl}-8-methoxy-4-oxo-1,4-dihydro-quinoline-3-carboxylic acid hydrochloride (Compound 7);
1-Cyclopropyl-6-fluoro-7-{3-[1-fluoro-2-(5-methyl-2-oxo-[1,3]dioxol-4-ylmethoxycarbonylamino)-ethylidene]-piperidin-1-yl}-8-methoxy-4-oxo-1,4-dihydro-quinoline-3-carboxylic acid (Compound 8);
1-Cyclopropyl-7-{3-[2-((2S)-2,6-diamino-hexanoylamino)-1-fluoro-ethylidene]-piperidin-1-yl}-6-fluoro-8-methoxy-4-oxo-1,4-dihydro-quinoline-3-carboxylic acid dihydrochloride (Compound 9);
7-(3-{2-[(2S)-2-((2S)-2-Amino-propionylamino)-4-methyl-pentanoylamino]-1-fluoro-ethylidene}-piperidin-1-yl)-1-cyclopropyl-6-fluoro-8-methoxy-4-oxo-1,4-dihydro-quinoline-3-carboxylic acid hydrochloride (Compound 10);
1-Cyclopropyl-7-{3-[2-(diethoxy-phosphorylamino)-1-fluoro-ethylidene]-piperidin-1-yl}-6-fluoro-8-methoxy-4-oxo-1,4-dihydro-quinoline-3-carboxylic acid sodium salt (Compound 13);
7-{3-[2-((2S)-2-Amino-3-dimethylamino-propionylamino)-1-fluoro-ethylidene]-piperidin-1-yl}-1-cyclopropyl-6-fluoro-8-methoxy-4-oxo-1,4-dihydro-quinoline-3-carboxylic acid dihydrochloride (Compound 14);
1-Cyclopropyl-7-[3-(2-{[(2S)-1-((2S)-2,6-diamino-hexanoyl)-pyrrolidine-2-carbonyl]-amino}-1-fluoro-ethylidene)-piperidin-1-yl]-6-fluoro-8-methoxy-4-oxo-1,4-dihydro-quinoline-3-carboxylic acid dihydrochloride (Compound 15);
7-(3-{2-[2-((2S)-2-Amino-4-carboxy-butyrylamino)-acetylamino]-1-fluoro-ethylidene}-piperidin-1-yl)-1-cyclopropyl-6-fluoro-8-methoxy-4-oxo-1,4-dihydro-quinoline-3-carboxylic acid hydrochloride (Compound 16); and

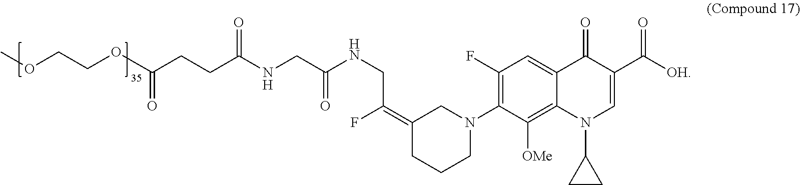

In another embodiment, the present invention is directed to a compound selected from the group consisting of 1-cyclopropyl-7-[3-(2-{[(2S)-1-((2S)-2,6-diamino-hexanoyl)-pyrrolidine-2-carbonyl]-amino}-1-fluoro-ethylidene)-piperidin-1-yl]-6-fluoro-8-methoxy-4-oxo-1,4-dihydro-quinoline-3-carboxylic acid (Compound 15) and pharmaceutically acceptable salts thereof, for example, the corresponding dihydrochloride salt.

In another embodiment, the present invention is directed to one or more compounds of formula (II) selected from the group consisting of
7-[3-(2-Amino-1-fluoro-ethylidene)-piperidin-1-yl]-1-cyclopropyl-6-fluoro-8-methoxy-4-oxo-1,4-dihydro-quinoline-3-carboxylic acid ethyl ester hydrochloride (Compound 11);
7-[3-(2-Amino-1-fluoro-ethylidene)-piperidin-1-yl]-1-cyclopropyl-6-fluoro-8-methoxy-4-oxo-1,4-dihydro-quinoline-3-carboxylic acid isopropyl ester hydrochloride (Compound 12);

and pharmaceutically acceptable salts thereof.

As used herein, the term “alkyl” whether used alone or as part of a substituent group, includes straight and branched chains. For example, alkyl radicals include methyl, ethyl, propyl, isopropyl, butyl, isobutyl, sec-butyl, t-butyl, pentyl and the like. Unless otherwise noted, “lower” when used with alkyl means a carbon chain composition of 1 to 4 carbon atoms. Thus, lower alkyl shall include methyl, ethyl, n-propyl, isopropyl, n-butyl, isobutyl, sec-butyl and t-butyl.

When a particular group is “substituted” (e.g., alkyl, cycloalkyl, aryl, heteroaryl, heterocycloalkyl, etc.), that group may have one or more substituents, preferably from one to five substituents, more preferably from one to three substituents, most preferably from one to two substituents, independently selected from the list of substituents.

With reference to substituents, the term “independently” means that when more than one of such substituents is possible, such substituents may be the same or different from each other.

As used herein, the notation “*” shall denote the presence of a stereogenic center. Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Preferably, wherein the compound is present as an enantiomer, the enantiomer is present at an enantiomeric excess of greater than or equal to about 80%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similarly, wherein the compound is present as a diastereomer, the diastereomer is present at a diastereomeric excess of greater than or equal to about 80%, more preferably, at a diastereomeric excess of greater than or equal to about 90%, more preferably still, at a diastereomeric excess of greater than or equal to about 95%, more preferably still, at a diastereomeric excess of greater than or equal to about 98%, most preferably, at a diastereomeric excess of greater than or equal to about 99%.

Furthermore, some of the crystalline forms for the compounds of the present invention may exist as polymorphs and as such are intended to be included in the present invention. In addition, some of the compounds of the present invention may form solvates with water (i.e., hydrates) or common organic solvents, and such solvates are also intended to be encompassed within the scope of this invention.

Abbreviations used in the specification, particularly the Schemes and Examples, are as follows:
Boc or BOC=tert-Butoxy-carbonyl
(Boc)₂O=Boc anhydride
BOP=Benzotriazol-1-yloxytris(dimethylamino)-phosphonium hexafluorophosphate
i-Bu=iso-Butyl
DBU=1,8-Diazabicyclo[5.4.0]undeca-7-ene
DCM=Dichloromethane
DIPEA or DIEA=Diisopropylethylamine
DMF=N,N-Dimethylformamide
DMSO=Dimethylsulfoxide
Et=Ethyl
Et₃N=Triethylamine
EtO=Ethoxy
EtOAc=Ethyl acetate
HATU=O-(7-Azabenzotriazol-1-yl)-N,N,N″,N″-Tetramethyl Uranium Hexafluorophosphate
HPLC=High Performance Liquid Chromatography
Me=Methyl
MeCN=Acetonitrile
MeO=Methoxy
MsO=Methanesulfonyloxy
NMP=N-methyl-2-pyrrolidinone
i-Pr₂NEt=Diisopropyl ethylamine
PyBrOP=Bromotri(pyrrolidino)phosphonium hexafluorophosphate
TEA=Triethylamine
TFA=Trifluoroacetic Acid
THF=Tetrahydrofuran

As used herein, unless otherwise noted, the term “isolated form” shall mean that the compound is present in a form which is separate from any solid mixture with another compound(s), solvent system or biological environment. In an embodiment of the present invention, the compound of formula (I) is present and/or prepared in an isolated form. In another embodiment of the present invention, the compound of formula (II) is present and/or prepared in an isolated form.

As used herein, unless otherwise noted, the term “substantially pure compound” shall mean that the mole percent of impurities in the isolated compound is less than about 5 mole percent, preferably less than about 2 mole percent, more preferably, less than about 0.5 mole percent, most preferably, less than about 0.1 mole percent. In an embodiment of the present invention, the compound of formula (I) is present and/or prepared as a substantially pure compound. In another embodiment of the present invention, the compound of formula (II) is present and/or prepared as a substantially pure compound.

As used herein, unless otherwise noted, the term “substantially free of a corresponding salt form(s)” when used to describe the compound of formula (I) or a compound of formula (II) shall mean that mole percent of the corresponding salt form(s) in the isolated base of formula (I) or compound of formula (II) is less than about 5 mole percent, preferably less than about 2 mole percent, more preferably, less than about 0.5 mole percent, most preferably less than about 0.1 mole percent. In an embodiment of the present invention, the compound of formula (I) is present and/or prepared as a compound which is substantially free of corresponding salt form(s). In another embodiment of the present invention, the compound of formula (II) is present and/or prepared as a compound which is substantially free of corresponding salt form(s).

The term “prophylactically effective amount” as used herein, means that amount of active compound or pharmaceutical agent that prevents the development of a condition, symptoms or manifestations thereof associated with bacterial infection. Thus it elicits the biological or medicinal response in a tissue system, animal or human that is being sought by a researcher, veterinarian, medical doctor or other clinician, which includes alleviation of the symptoms of the disease or disorder being treated.

The term “drug-resistant” or “drug-resistance” refers to the characteristics of a microbe to survive in the presence of a currently available antimicrobial agent such as an antibiotic at its routine, effective concentration.

As used herein, unless otherwise noted, the terms “treating”, “treatment” and the like, shall include the management and care of a subject or patient (preferably mammal, more preferably human) for the purpose of combating a disease, condition, or disorder and includes the administration of a compound of the present invention to prevent the onset of the symptoms or complications, alleviate the symptoms or complications, or eliminate the disease, condition, or disorder.

As used herein, unless otherwise noted, the term “prevention” shall include (a) reduction in the frequency of one or more symptoms; (b) reduction in the severity of one or more symptoms; (c) the delay or avoidance of the development of additional symptoms; and/or (d) delay or avoidance of the development of the disorder or condition.

One skilled in the art will recognize that wherein the present invention is directed to methods of prevention, a subject in need of thereof (i.e. a subject in need of prevention) shall include any subject or patient (preferably a mammal, more preferably a human) who has experienced or exhibited at least one symptom of the disorder, disease or condition to be prevented. Further, a subject in need thereof may additionally be a subject (preferably a mammal, more preferably a human) who has not exhibited any symptoms of the disorder, disease or condition to be prevented, but who has been deemed by a physician, clinician or other medical professional to be at risk of developing said disorder, disease or condition. For example, the subject may be deemed at risk of developing a disorder, disease or condition (and therefore in need of prevention or preventive treatment) as a consequence of the subject's medical history, including, but not limited to, family history, pre-disposition, co-existing (comorbid) disorders or conditions, genetic testing, and the like.

The term “subject” as used herein, refers to an animal, preferably a mammal, most preferably a human, who has been the object of treatment, observation or experiment. Preferably, the subject has experienced and/or exhibited at least one symptom of the disease or disorder to be treated and/or prevented.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

As more extensively provided in this written description, terms such as “reacting” and “reacted” are used herein in reference to a chemical entity that is any one of: (a) the actually recited form of such chemical entity, and (b) any of the forms of such chemical entity in the medium in which the compound is being considered when named.

One skilled in the art will recognize that, where not otherwise specified, the reaction step(s) is performed under suitable conditions, according to known methods, to provide the desired product. One skilled in the art will further recognize that, in the specification and claims as presented herein, wherein a reagent or reagent class/type (e.g. base, solvent, etc.) is recited in more than one step of a process, the individual reagents are independently selected for each reaction step and may be the same of different from each other. For example wherein two steps of a process recite an organic or inorganic base as a reagent, the organic or inorganic base selected for the first step may be the same or different than the organic or inorganic base of the second step. Further, one skilled in the art will recognize that wherein a reaction step of the present invention may be carried out in a variety of solvents or solvent systems, said reaction step may also be carried out in a mixture of the suitable solvents or solvent systems.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any amount or range therein.

Examples of suitable solvents, bases, reaction temperatures, and other reaction parameters and components are provided in the detailed description which follows herein. One skilled in the art will recognize that the listing of said examples is not intended, and should not be construed, as limiting in any way the invention set forth in the claims which follow thereafter.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

As used herein, unless otherwise noted, the term “nitrogen protecting group” shall mean a group which may be attached to a nitrogen atom to protect said nitrogen atom from participating in a reaction and which may be readily removed following the reaction. Suitable nitrogen protecting groups include, but are not limited to carbamates—groups of the formula —C(O)O—R wherein R is for example methyl, ethyl, t-butyl, benzyl, phenylethyl, CH₂═CH—CH₂—, and the like; amides—groups of the formula —C(O)—R′ wherein R′ is for example methyl, phenyl, trifluoromethyl, and the like; N-sulfonyl derivatives—groups of the formula —SO₂—R″ wherein R″ is for example tolyl, phenyl, trifluoromethyl, 2,2,5,7,8-pentamethylchroman-6-yl-, 2,3,6-trimethyl-4-methoxybenzene, and the like. Other suitable nitrogen protecting groups may be found in texts such as T. W. Greene & P. G. M. Wuts, Protective Groups in Organic Synthesis, John Wiley & Sons, 1991.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

Additionally, chiral HPLC against a standard may be used to determine percent enantiomeric excess (% ee). The enantiomeric excess may be calculated as follows

[(Rmoles−Smoles)/(Rmoles+Smoles)]×100%

where Rmoles and Smoles are the R and S mole fractions in the mixture such that Rmoles+Smoles=1. The enantiomeric excess may alternatively be calculated from the specific rotations of the desired enantiomer and the prepared mixture as follows:

ee=([α−obs]/[α−max])×100.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include, but are not limited to, the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids which may be used in the preparation of pharmaceutically acceptable salts include, but are not limited to, the following: acids including acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid.

Representative bases which may be used in the preparation of pharmaceutically acceptable salts include, but are not limited to, the following: bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, sodium hydroxide, triethanolamine, tromethamine and zinc hydroxide.

Compounds of formula (II) may be prepared according to the process outlined in Scheme 1.

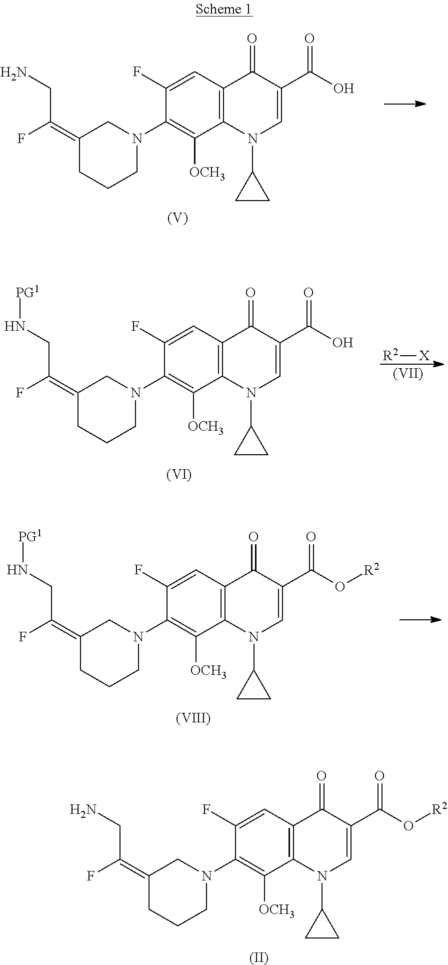

Accordingly, a compound of formula (V), also known as (E)-7-(3-(2-amino-1-fluoroethylidene)piperidin-1-yl)-1-cyclopropyl-6-fluoro-8-methoxy-4-oxo-1,4-dihydroquinoline-3-carboxylic acid, a known compound is protected according to known methods, to yield the corresponding compound of formula (VI), wherein PG¹ is the corresponding nitrogen protecting group. For example, the compound of formula (V) may be reacted with BOC anhydride, in the presence of an organic base such as TEA, DIPEA, and the like, in a suitably selected organic solvent such as THF, DCM, and the like, to yield the corresponding compound of formula (VI), wherein PG¹ is BOC.

The compound of formula (VI) is reacted with a suitably substituted compound of formula (VII), wherein X is Br, I, OMs, and the like, a known compound or compound prepared by known methods; in the presence of a suitably selected organic or inorganic base such as Cs₂CO₃, K₂CO₃, DBU, and the like; in a suitably selected organic solvent such as acetonitrile, NMP, and the like, to yield the corresponding compound of formula (VIII).

The compound of formula (VIII) is de-protected according to known methods, to yield the corresponding compound of formula (II). For example, wherein PG¹ is BOC, the compound of formula (VIII) may be de-protected by reacting with a suitably selected acid such as HCl, TFA, and the like; in a suitably selected organic solvent such as DCM, 1,4-dioxane, and the like, or mixture of said solvents such as a mixture of 1,4-dioxane and DCM.

Compounds of formula (I) wherein R¹ is —P(O)(OR⁷)₂ may be prepared according to the process outlined in Scheme 2 below.

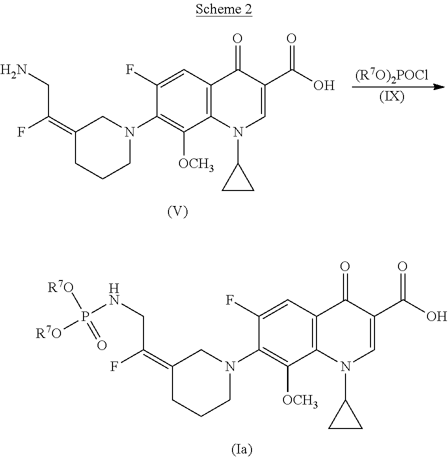

Accordingly, a compound of formula (V), a known compound is reacted with a suitably substituted compound of formula (IX), a known compound or compound prepared by known methods; in the presence of a suitably selected organic base such as TEA, DIPEA, and the like; in a suitably selected organic solvent such as THF, methylene chloride, 1,4-dioxane, and the like; to yield the corresponding compound of formula (Ia).

Compounds of formula (I) wherein R¹ is may be

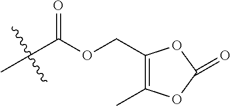

prepared according to the process outlined in Scheme 3.

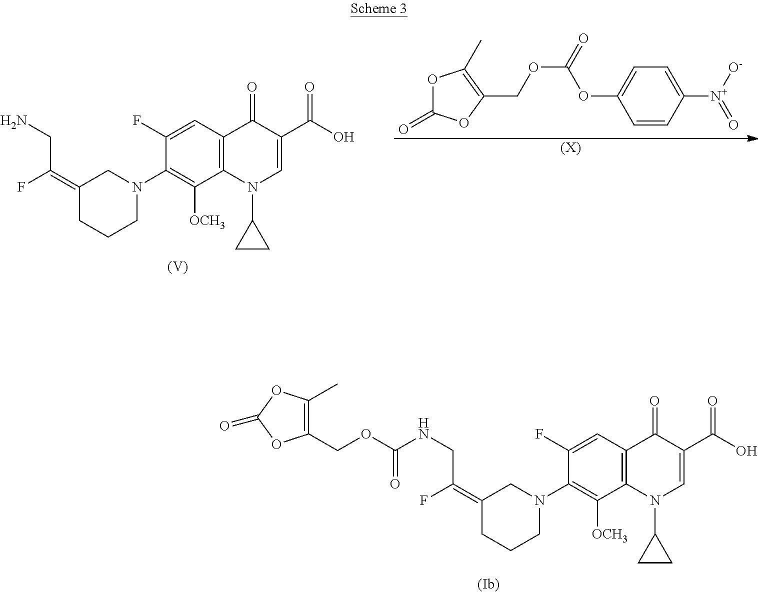

Accordingly, a compound of formula (V), a known compound is reacted with a compound of formula (X), also known as carbonic acid 5-methyl-2-oxo-[1,3]dioxol-4-ylmethyl ester 4-nitro-phenyl ester, a known compound or compound prepared by known methods, in the presence of a suitably selected organic base such as TEA, DIPEA, and the like; in a suitably selected organic solvent such as THF, methylene chloride, DMF, and the like; to yield the corresponding compound of formula (Ib).

Compounds of formula (I) wherein R¹ is

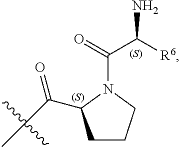

may be prepared according to the process outlined in Scheme 4 below.

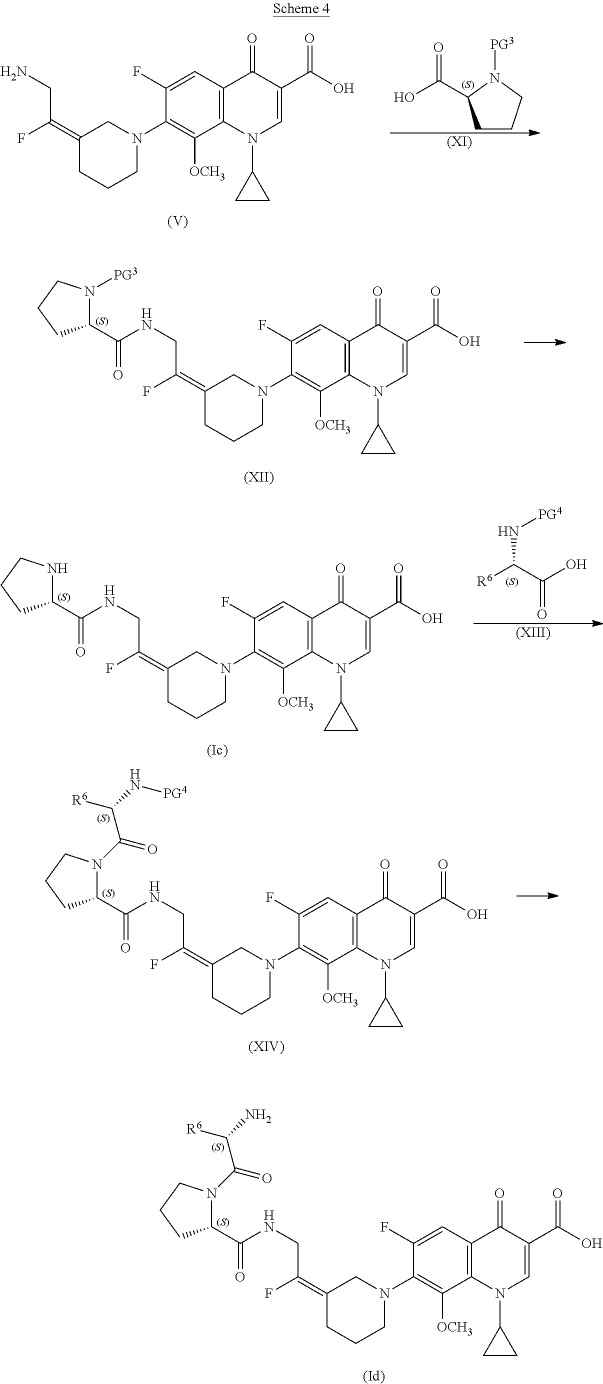

Accordingly, a compound of formula (V), a known compound is reacted with a compound of formula (XI), wherein PG³ is a suitably selected nitrogen protecting group such as BOC, and the like, a known compound or compound prepared by known methods; in the presence of a suitably selected coupling agent such as HATU, BOP, PyBrOP, and the like; in the presence of an organic base such as TEA, DIPEA, pyridine, and the like; in a suitably selected organic solvent such as THF, methylene chloride, DMF, and the like; to yield the corresponding compound of formula (XII).

The compound of formula (XII) is de-protected according to known methods, to yield the corresponding compound of formula (Ic). For example, wherein the compound of formula (XII), PG³ is BOC, the compound may be de-protected by reacting with a suitably selected acid such as HCl, TFA, and the like; in a suitably selected organic solvent such as 1,4-dioxane, DCM, and the like.

The compound of formula (Ic) may be further reacted with a suitably substituted compound of formula (XIII), wherein PG⁴ is a suitably selected nitrogen protecting group such as BOC, and the like; in the presence of a suitably selected coupling agent such as HATU, BOP, PyBrOP, and the like; in the presence of an organic base such as TEA, DIPEA, pyridine, and the like; in a suitably selected organic solvent such as THF, methylene chloride, DMF, and the like; to yield the corresponding compound of formula (XIV).

The compound of formula (XIV) is de-protected according to known methods, to yield the corresponding compound of formula (Id).

One skilled in the art will recognize that wherein the compound of formula (XIV), the R⁶ group contains a reactive group (for example, an amino group), said reactive group is preferably protected prior to the reaction of the compound of formula (XIV) with the compound formula (Ic). Said protecting group is then removed, according to known methods, either simultaneously with the removal of the PG⁴ protecting group or in a separate reaction step, under orthogonal reaction conditions.

Compounds of formula (I) wherein R¹ is —C(O)—CH(R³)—NR⁴R⁵, wherein R³, R⁴ are as herein defined and wherein R⁵ is selected from the group consisting of hydrogen, —C(O)—CH(CH₃)—NH₂, —C(O)—CH[(CH₂)₄NH₂]—NH₂ and —C(O)—CH[(CH₂)₂CO₂H]—NH₂, may be prepared according to the process outlined in Scheme 5 below.

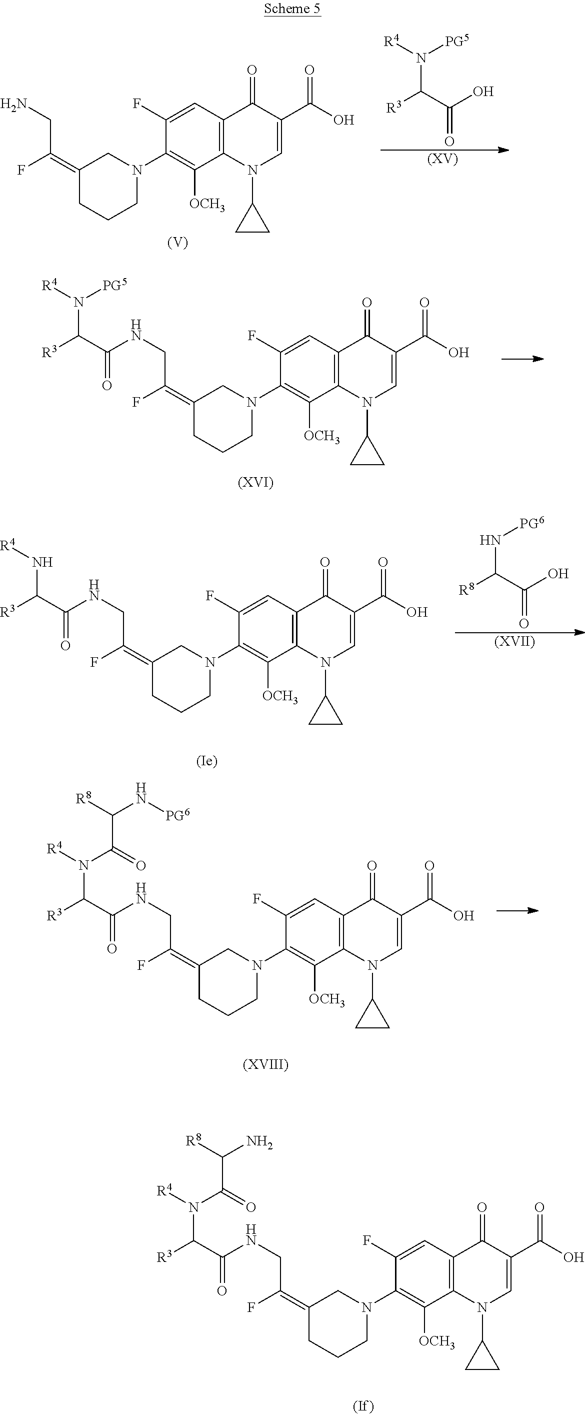

Accordingly, a compound of formula (V), a known compound, is reacted with a suitably substituted compound of formula (XV), wherein PG⁵ is a suitably selected nitrogen protecting group such as BOC, and the like; in the presence of a suitably selected coupling agent such as HATU, BOP, PyBrOP, and the like; in the presence of an organic base such as TEA, DIPEA, pyridine, and the like; in a suitably selected organic solvent such as THF, methylene chloride, DMF, and the like to yield the corresponding compound of formula (XVI).

Alternatively, a compound of formula (V), a known compound, is reacted with a suitably substituted compound of formula (XV), wherein PG⁵ is a suitably selected nitrogen protecting group such as BOC, and the like; in the presence of a suitably selected chloroformate of the formula Cl—C(O)—O-(lower alkyl); in the presence of an organic base such as TEA, DIPEA, pyridine, and the like; to yield the corresponding compound of formula (XVI).

The compound of formula (XVI) is de-protected according to known methods, to yield the corresponding compound of formula (Ie). For example, wherein the compound of formula (XVI), PG⁵ is BOC, the compound may be de-protected by reacting with a suitably selected acid such as HCl, TFA, and the like; in a suitably selected organic solvent such as 1,4-dioxane, DCM, and the like.

One skilled in the art will recognize that wherein the compound of formula (XV), the R³ group contains a reactive group (for example, an amino group), said reactive group(s) are preferably protected prior to the reaction of the compound of formula (XV) with the compound formula (V). Said protecting group(s) are then removed, according to known methods, either simultaneously with the removal of the PG⁵ protecting group or in one or more separate reaction steps, under orthogonal reaction conditions.

The compound of formula (Ie) is further, optionally reacted with a suitably substituted compound of formula (XVII), wherein R⁸ is selected from the group consisting of —CH₃, —(CH₂)₄NH-PG⁷ and —(CH₂)₂CO₂—PG⁸, wherein PG⁶ and PG⁷ are each a suitable, independently selected, nitrogen protecting group such as BOC, and the like, and wherein PG⁸ is a suitably selected carboxylic acid protecting group such as tert-butyl, and the like, a known compound or compound prepared by known methods; in the presence of a suitably selected coupling agent such as HATU, BOP, PyBrOP, and the like; in the presence of a suitably selected organic base such as TEA, DIPEA, pyridine, and the like; in a suitably selected organic solvent such as THF, methylene chloride, DMF, and the like; to yield the corresponding compound of formula (XVIII).

Alternatively, the compound of formula (Ie) is reacted with a suitably substituted compound of formula (XVII), wherein R⁸ is selected from the group consisting of —CH₃, —(CH₂)₄NH-PG⁷ and —(CH₂)₂CO₂—PG⁸, wherein PG⁶ and PG⁷ are each a suitable, independently selected nitrogen protecting group such as BOC, and the like, and wherein PG⁸ is a suitably selected carboxylic acid protecting group such as tert-butyl, and the like, a known compound or compound prepared by known methods; in the presence of a suitably selected chloroformate of the formula Cl—C(O)—O-(lower alkyl), wherein the lower alkyl is preferably ethyl or isobutyl; in the presence of a suitably selected organic base such as TEA, DIPEA, pyridine, and the like; to yield the corresponding compound of formula (XVIII).

The compound of formula (XVIII) is then de-protected, according to known methods, for example as described above for previous de-protection steps, to yield the corresponding compound of formula (If).

One skilled in the art will recognize that the PG⁶, PG⁷, and PG⁸ protecting groups may be selected so as to be removed, according to known methods, either simultaneously or in one or more separate reaction steps, under orthogonal reaction conditions.

Alternatively, compounds of formula (I) wherein R¹ is —C(O)—CH(R³)—NR⁴R⁵, wherein R³ and R⁴ are as herein defined and wherein R⁵ is selected from the group consisting of —C(O)—CH(CH₃)—NH₂, —C(O)—CH[(CH₂)₄NH₂]—NH₂ and —C(O)—CH[(CH₂)₂CO₂H]—NH₂, may be prepared according to the process outlined in Scheme 6, below.

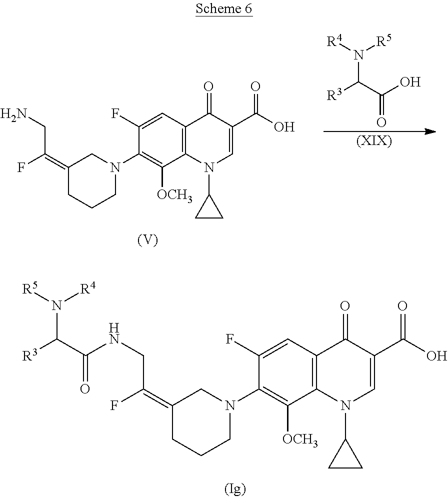

Accordingly, a suitably substituted compound of formula (V), a known compound, is reacted with a suitably substituted compound of formula (XIX), a known compound or compound prepared by known methods; in the presence of a suitably selected coupling agent such as HATU, BOP, PyBrOP, and the like; in the presence of an organic base such as TEA, DIPEA, pyridine, and the like; in a suitably selected organic solvent such as THF, methylene chloride, DMF, and the like; to yield the corresponding compound of formula (Ig).

One skilled in the art will recognize that wherein the compound of formula (XVII), the R³ and/or the R⁵ group(s) contain a reactive group (for example, an amino group), said reactive group(s) are preferably protected prior to the reaction of the compound of formula (XIX) with the compound of formula (V). Said protecting group(s) are then removed, according to known methods, either simultaneously or in one or more separate reaction steps, under orthogonal reaction conditions.

The compound of formula (I) wherein R¹ is —C(O)—CH₂—NH—C(O)—(CH₂)₂—C(O)-mPEG(2000) may be prepared according to the process as described in Scheme 7, below.

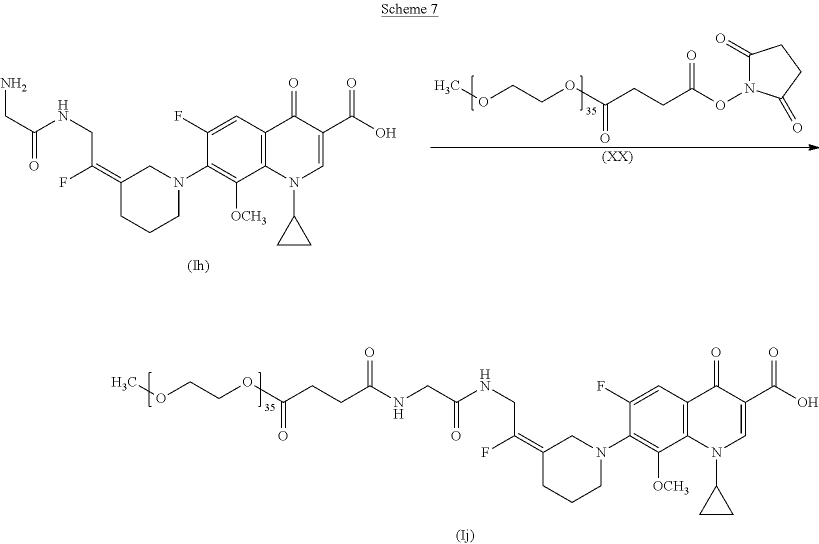

Accordingly, a compound of formula (Ih), a compound of formula (I) wherein R¹ is —C(O)—CH₂—NH₂, prepared as described herein, is reacted with a suitably substituted compound of formula (XX), a known compound or compound prepared by known methods; in the presence of a suitably selected organic base such as TEA, DIPEA, pyridine, and the like; in a suitably selected organic solvent such as DMF, THF, methylene chloride, and the like; to yield the corresponding compound of formula (Ij).

The present invention further comprises pharmaceutical compositions containing one or more compounds of formula (I) and/or one or more compounds of formula (II) with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending on the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gel caps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, though other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.01 to about 1000 mg or any amount or range therein, and may be given at a dosage of from about 0.01 to about 100 mg/kg/day, or any amount or range therein, preferably from about 0.1 to about 50 mg/kg/day, or any amount or range therein, more preferably from about 0.5 to about 25 mg/kg/day, or any amount or range therein, more preferably from about 1.0 to about 10.0 mg/kg/day, or any amount or range therein. The dosages, however, may be varied depending upon the requirement of the patients, the severity of the condition being treated and the compound being employed. The use of either daily administration or post-periodic dosing may be employed.

Preferably these compositions are in unit dosage forms such as tablets, pills, capsules, powders, granules, sterile parenteral solutions or suspensions, metered aerosol or liquid sprays, drops, ampoules, autoinjector devices or suppositories; for oral, parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.01 to about 1,000 mg, or any amount or range therein, of the active ingredient of the present invention. The tablets or pills of the novel composition can be coated or otherwise compounded to provide a dosage form yielding the advantage of prolonged action. For example, the tablet or pill can comprise an inner dosage and an outer dosage component, the latter being in the form of an envelope over the former. The two components can be separated by an enteric layer, which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The methods described in the present invention may also be carried out using a pharmaceutical composition comprising any of the compounds as defined herein and a pharmaceutically acceptable carrier. The pharmaceutical composition may contain between about 0.01 mg and 1,000 mg of the compound, or any amount or range therein; preferably about 10 to 500 mg of the compound, or any amount or range therein, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds of the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms may include suitably flavored suspending or dispersing agents such as the synthetic and natural gums, for example, tragacanth, acacia, methyl-cellulose and the like. For parenteral administration, sterile suspensions and solutions are desired. Isotonic preparations, which generally contain suitable preservatives, are employed when intravenous administration is desired.

The compound of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine or phosphatidylcholine.

Compounds of the present invention may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamidephenol, polyhydroxy-ethylaspartamidephenol, or polyethyleneoxidepolylysine substituted with palmitoyl residue. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

To prepare a pharmaceutical composition of the present invention, a compound of formula (I) or a compound of formula (II) as the active ingredient is intimately admixed with a pharmaceutical carrier according to conventional pharmaceutical compounding techniques, which carrier may take a wide variety of forms depending on the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms Parenteral Medications, Volumes 1-2, edited by Avis et al; and Pharmaceutical Dosage Forms: Disperse Systems, Volumes 1-2, edited by Lieberman et al; published by Marcel Dekker, Inc.

Compounds of this invention may be administered in any of the foregoing compositions and according to dosage regimens established in the art whenever treatment with antimicrobial agents is required.

The daily dosage of the products may be varied over a wide range from 0.01 to 10,000 mg per adult human per day, or any amount or range therein. For oral administration, the compositions are preferably provided in the form of tablets containing about 0.01, 0.05, 0.1, 0.5, 1.0, 2.5, 5.0, 10.0, 15.0, 25.0, 50.0, 100, 150, 200, 250, 500, 750 and 1000 milligrams of the active ingredient for the symptomatic adjustment of the dosage to the patient to be treated. An effective amount of the drug is ordinarily supplied at a dosage level of from about 0.01 mg/kg to about 100 mg/kg of body weight per day, or any amount or range therein. Preferably, the range is from about 0.1 to about 50 mg/kg of body weight per day, or any amount or range therein. More preferably, from about 0.5 to about 25 mg/kg of body weight per day, or any amount or range therein. More preferably, from about 1.0 to about 10 mg/kg of body weight per day, or any amount or range therein. The compounds may be administered on a regimen of 1 to 4 times per day.

Optimal dosages to be administered may be readily determined by those skilled in the art, and will vary with the particular compound used, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder. One skilled in the art will further recognize that human clinical trails including first-in-human, dose in the range of and efficacy trials, in healthy subjects and/or those suffering from a given disorder, may be completed according to methods well known in the clinical and medical arts.

The following Examples are set forth to aid in the understanding of the invention, and are not intended and should not be construed to limit in any way the invention set forth in the claims which follow thereafter.

In the Examples that follow, some synthesis products are listed as having been isolated as a “residue”. It will be understood by one of ordinary skill in the art that the term “residue” does not limit the physical state in which the product was isolated and may include, for example, a solid, an oil, a foam, a gum, a syrup, and the like.

### Example 1

### Compound #2

### 7-(3-{2-[(2S)-2-(2S)-2-Amino-propionylamino)-propionylamino]-1-fluoro-ethylidene}-piperidin-1-yl)-1-cyclopropyl-6-fluoro-8-methoxy-4-oxo-1,4-dihydro-quinoline-3-carboxylic acid hydrochloride

### STEP A: 7-(3-{2-[(25)-2-((2S)-2-tert-Butoxycarbonylamino-propionylamino)-propionylamino]-1-fluoro-ethylidene}-piperidin-1-yl)-1-cyclopropyl-6-fluoro-8-methoxy-4-oxo-1,4-dihydro-quinoline-3-carboxylic acid

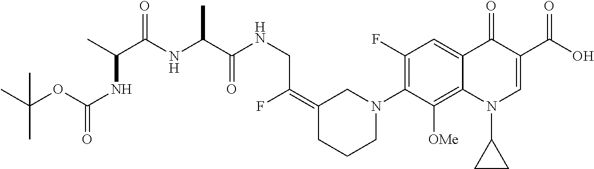

Neat ClCO₂CH₂CH₃ (1.77 mL, 18.5 mmol) was added to a THF solution (200 mL) of Boc-L-Ala-L-Ala (5.0 g, 19.4 mmol) and i-Pr₂NEt (9.2 mL, 52.8 mmol). After 1 h solid 7-[3-(2-amino-1-fluoro-ethylidene)-piperidin-1-yl]-1-cyclopropyl-6-fluoro-8-methoxy-4-oxo-1,4-dihydro-quinoline-3-carboxylic acid (8.0 g, 17.6 mmol) was added to the cloudy mixture. After 16 h, the resulting homogeneous solution was diluted with EtOAc and washed with water and brine, then dried (Na₂SO₄), concentrated and purified via column chromatography to yield a yellow gum.

¹H NMR (DMF-d₇, 300 MHz): δ=9.12 (s, 1H), 8.92-9.07 (m, 1H), 8.79-8.92 (m, 1H), 8.16 (dd, J=12.1, 2.3 Hz, 1H), 4.65-4.78 (m, 1H), 4.53-4.64 (m, 1 H), 4.50 (br. s., 1H), 4.31-4.46 (m, 3H), 4.17-4.28 (m, 2H), 4.08-4.17 (m, 4H), 3.83 (br. s., 2H), 2.79 (br. s., 2H), 2.13 (br. s., 2H), 1.71 (dd, J=17.0, 7.2 Hz, 3H), 1.50-1.63 (m, 5H), 1.45 ppm (d, J=6.0 Hz, 9H).

### STEP B: 7-(3-{2-[(2S)-2-((2S)-2-Amino-propionylamino)-propionylamino]-1-fluoro-ethylidene}-piperidin-1-yl)-1-cyclopropyl-6-fluoro-8-methoxy-4-oxo-1,4-dihydro-quinoline-3-carboxylic acid hydrochloride

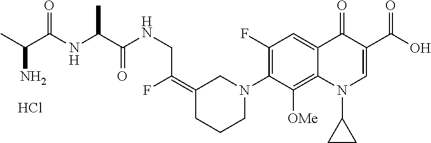

A 4 M HCl solution in dioxane (70 mL, 278.4 mmol) was added to a CH₂Cl₂ solution (70 mL) of 7-(3-{2-[2-((2S)-2-tert-butoxycarbonylamino-propionylamino)-propionylamino]-1-fluoro-ethylidene}-piperidin-1-yl)-1-cyclopropyl-6-fluoro-8-methoxy-4-oxo-1,4-dihydro-quinoline-3-carboxylic acid (9.2 g, 13.9 mmol). After 2 h, the liquid was decanted off of the residue on the bottom of the flask. The residue was washed with CH₂Cl₂ (3×) and then dried in vacuo to yield the title compound as a yellow solid.

¹H NMR (DMSO-d₆, 300 MHz): δ=14.96 (s, 1H), 8.71 (s, 1H), 8.65 (d, J=7.5 Hz, 1H), 8.38-8.51 (m, 1H), 8.15 (br. s., 3H), 7.76 (dd, J=12.4, 1.9 Hz, 1H), 4.23-4.40 (m, 1H), 4.18 (br. s., 1H), 3.92-4.10 (m, 4H), 3.83 (br. s., 1H), 3.74 (d, J=2.3 Hz, 3H), 3.38-3.48 (m, 2H), 2.30-2.44 (m, 2H), 1.73 (br. s., 2H), 1.29 (dd, J=15.6, 7.0 Hz, 3H), 1.09-1.22 (m, 4H), 0.96-1.09 ppm (m, 3H); MS m/z 562 (M+H).

### Example 2

### Compound #6

### 7-{3-[2-(2-Amino-acetylamino)-1-fluoro-ethylidene]-piperidin-1-yl}-1-cyclopropyl-6-fluoro-8-methoxy-4-oxo-1,4-dihydro-quinoline-3-carboxylic acid hydrochloride

### STEP A: 7-{3-[2-(2-tert-Butoxycarbonylamino-acetylamino)-1-fluoro-ethylidene]-piperidin-1-yl}-1-cyclopropyl-6-fluoro-8-methoxy-4-oxo-1,4-dihydro-quinoline-3-carboxylic acid

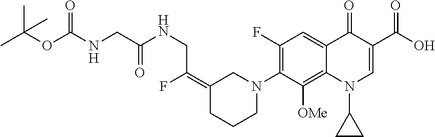

Neat ClCO₂CH₂CH₃ (1.77 mL, 18.5 mmol) was added to a THF solution (200 mL) of Boc-Gly (3.4 g, 19.3 mmol) and i-Pr₂NEt (9.2 mL, 52.8 mmol). After 1 h, solid 7-[3-(2-amino-1-fluoro-ethylidene)-piperidin-1-yl]-1-cyclopropyl-6-fluoro-8-methoxy-4-oxo-1,4-dihydro-quinoline-3-carboxylic acid (8.0 g, 17.6 mmol) was added to the cloudy mixture. After 16 h, the resulting homogeneous solution was diluted with EtOAc and washed with water and brine, then dried (Na₂SO₄), concentrated and purified via column chromatography to yield a yellow gum.

¹H NMR (CHLOROFORM-d, 300 MHz): δ=14.87 (s, 1H), 8.78 (s, 1H), 7.76 (d, J=12.4 Hz, 1H), 6.91 (br. s., 1H), 5.31-5.39 (m, 1H), 4.13-4.27 (m, 2H), 4.08 (tt, J=7.3, 3.7 Hz, 1H), 4.00 (s, 2H), 3.79 (s, 3H), 3.76-3.83 (m, 2H), 3.47 (br. s., 2 H), 2.45 (t, J=5.3 Hz, 2H), 1.80 (br. s., 2H), 1.43 (s, 9H), 1.23-1.33 (m, 2H), 0.98-1.10 ppm (m, 2H)

### STEP B: 7-{3-[2-(2-Amino-acetylamino)-1-fluoro-ethylidene]-piperidin-1-yl}-1-cyclopropyl-6-fluoro-8-methoxy-4-oxo-1,4-dihydro-quinoline-3-carboxylic acid hydrochloride

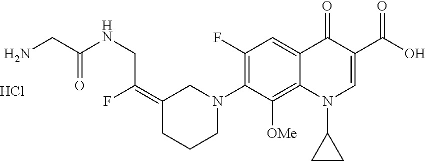

A 4 M HCl solution in dioxane (63 mL, 253.5 mmol) was added to a CH₂Cl₂ solution (63 mL) of 7-{3-[2-(2-tert-butoxycarbonylamino-acetylamino)-1-fluoro-ethylidene]-piperidin-1-yl}-1-cyclopropyl-6-fluoro-8-methoxy-4-oxo-1,4-dihydro-quinoline-3-carboxylic acid (7.3 g, 12.7 mmol). After 2 h the liquid was decanted off of the residue on the bottom of the flask. The residue was washed with CH₂Cl₂ (3×) and then dried in vacuo to yield the title compound as a yellow solid.

¹H NMR (DMSO-d₆, 300 MHz): δ=14.95 (br. s., 1H), 8.87 (t, J=5.1 Hz, 1H), 8.71 (s, 1H), 8.14 (br. s., 3H), 7.76 (d, J=12.1 Hz, 1H), 4.13-4.27 (m, 2H), 4.08 (d, J=5.3 Hz, 1H), 3.97 (s, 2H), 3.75 (s, 3H), 3.49-3.56 (m, 1H), 3.43 (br. s., 2H), 3.32-3.40 (m, 1H), 2.39 (br. s., 2H), 1.73 (br. s., 2H), 1.09-1.21 (m, 2H), 0.98-1.09 ppm (m, 2H); MS m/z 477 (M+H).

### Example 3

### Compound #9

### 1-Cyclopropyl-7-{3-[2-((2S)-2,6-diamino-hexanoylamino)-1-fluoro-ethylidene}-piperidin-1-yl]-6-fluoro-8-methoxy-4-oxo-1,4-dihydro-quinoline-3-carboxylic acid dihydrochloride

### STEP A: 7-{3-[2-((2S)-2,6-Bis-tert-butoxycarbonylamino-hexanoylamino)-1-fluoro-ethylidene]-piperidin-1-yl}-1-cyclopropyl-6-fluoro-8-methoxy-4-oxo-1,4-dihydro-quinoline-3-carboxylic acid

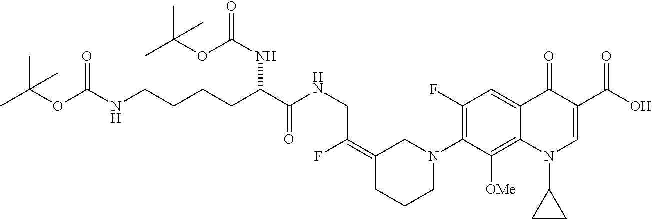

Neat ClCO₂i-Bu (2.4 mL, 18.4 mmol) was added to a THF solution (200 mL) of (S)-2,6-bis-tert-butoxycarbonylamino-hexanoic acid (3.4 g, 19.3 mmol) and i-Pr₂NEt (9.1 mL, 52.6 mmol). After 1 h, solid 7-[3-(2-amino-1-fluoro-ethylidene)-piperidin-1-yl]-1-cyclopropyl-6-fluoro-8-methoxy-4-oxo-1,4-dihydro-quinoline-3-carboxylic acid (8.0 g, 17.6 mmol) was added to the cloudy mixture. After 16 h, the resulting homogeneous solution was diluted with EtOAc and washed with water and brine, then dried (Na₂SO₄), concentrated and purified via column chromatography to yield a yellow gum.

MS m/z 734 (M+H).

### STEP B: 1-Cyclopropyl-7-{3-[2-((2S)-2,6-diamino-hexanoylamino)-1-fluoro-ethylidene]-piperidin-1-yl}-6-fluoro-8-methoxy-4-oxo-1,4-dihydro-quinoline-3-carboxylic acid dihydrochloride

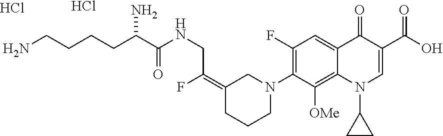

A 4 M HCl solution in dioxane (31 mL, 123 mmol) was added to a CH₂Cl₂ solution (100 mL) of 7-{3-[2-((2S)-2,6-bis-tert-butoxycarbonylamino-hexanoylamino)-1-fluoro-ethylidene]-piperidin-1-yl}-1-cyclopropyl-6-fluoro-8-methoxy-4-oxo-1,4-dihydro-quinoline-3-carboxylic acid (9.0 g, 12.3 mmol). After 2 h, the liquid was decanted off of the residue on the bottom of the flask. The residue was washed with CH₂Cl₂ (3×) and then dried in vacuo to yield the title compound as a yellow solid.

¹H NMR (300 MHz, CHLOROFORM-d) δ 14.93 (s, 1H), 8.82 (s, 1H), 7.86 (d, J=12.06 Hz, 1H), 6.58 (br. s., 1H), 5.07 (br. s., 1H), 4.62 (br. s., 1H), 4.19 (d, J=5.65 Hz, 1H), 4.03-4.15 (m, 2H), 3.98 (s, 3H), 3.42-3.52 (m, 2H), 3.19 (qd, J=4.52, 7.41 Hz, 2H), 3.02-3.14 (m, 2H), 2.45 (br. s., 2H), 1.73-1.87 (m, 2H), 1.68 (s, 4H), 1.33-1.56 (m, 4H), 1.21-1.30 (m, 2H), 0.96-1.07 (m, 2H); MS m/z 548 (M+H).

### Example 4

### Compound #1

### 7-{3-[2-((2S)-2-Amino-3-methyl-butyrylamino)-1-fluoro-ethylidene]-piperidin-1-yl}-1-cyclopropyl-6-fluoro-8-methoxy-4-oxo-1,4-dihydro-quinoline-3-carboxylic acid hydrochloride

### STEP A: 7-{3-[2-((2S)-2-tert-Butoxycarbonylamino-3-methyl-butyrylamino)-1-fluoro-ethylidene]-piperidin-1-yl}-1-cyclopropyl-6-fluoro-8-methoxy-4-oxo-1,4-dihydro-quinoline-3-carboxylic acid

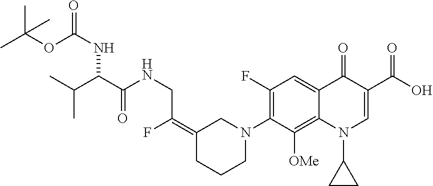

Neat ClCO₂CH₂CH₃ (1.8 mL, 18.4 mmol) was added to a THF solution (200 mL) of (S)-2-tert-butoxycarbonylamino-3-methyl-butyric acid (4.6 g, 21.1 mmol) and 1-Pr₂NEt (9.1 mL, 52.6 mmol). After 1 h, solid 7-[3-(2-amino-1-fluoro-ethylidene)-piperidin-1-yl]-1-cyclopropyl-6-fluoro-8-methoxy-4-oxo-1,4-dihydro-quinoline-3-carboxylic acid (8.0 g, 17.6 mmol) was added to the cloudy mixture. After 16 h, the resulting homogeneous solution was diluted with EtOAc and washed with water and brine, then dried (Na₂SO₄), concentrated and purified via column chromatography to yield a yellow gum.

MS m/z 619 (M+H).

### STEP B: 7-{3-[2-((2S)-2-Amino-3-methyl-butyrylamino)-1-fluoro-ethylidene]-piperidin-1-yl}-1-cyclopropyl-6-fluoro-8-methoxy-4-oxo-1,4-dihydro-quinoline-3-carboxylic acid hydrochloride

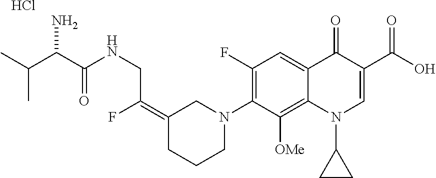

A 4 M HCl solution in dioxane (38 mL, 152 mmol) was added to a CH₂Cl₂ solution (100 mL) of 7-{3-[2-((2S)-2-tert-butoxycarbonylamino-3-methyl-butyrylamino)-1-fluoro-ethylidene]-piperidin-1-yl}-1-cyclopropyl-6-fluoro-8-methoxy-4-oxo-1,4-dihydro-quinoline-3-carboxylic acid (9.5 g, 15.4 mmol). After 2 h, the liquid was decanted off of the residue on the bottom of the flask. The residue was washed with CH₂Cl₂ (3×) and then dried in vacuo to yield the title compound as a yellow solid.

¹H NMR (300 MHz, CHLOROFORM-d) δ 14.79 (s, 1H), 8.82 (s, 1H), 7.79-7.98 (m, 1H), 6.26 (br. s., 1H), 4.92 (br. s., 1H), 4.20 (dd, J=6.03, 9.80 Hz, 1H), 4.09-4.17 (m, 2H), 4.01-4.09 (m, 2H), 3.95-4.01 (m, 2H), 3.85 (dd, J=6.22, 8.48 Hz, 1H), 3.73-3.78 (m, 3H), 3.40-3.52 (m, 2H), 2.46 (br. s., 2H), 1.74-1.86 (m, 2H), 1.34-1.49 (m, 3H), 1.17-1.30 (m, 3H), 0.97-1.08 (m, 4H); MS m/z 519 (M+H).

### Example 5

### Compound #3

### 7-{3-[2-((2S)-2-Amino-3-phenyl-propionylamino)-1-fluoro-ethylidene]-piperidin-1-yl}-1-cyclopropyl-6-fluoro-8-methoxy-4-oxo-1,4-dihydro-quinoline-3-carboxylic acid hydrochloride

### STEP A: 7-{3-[2-((2S)-2-tert-Butoxycarbonylamino-3-phenyl-propionylamino)-1-fluoro-ethylidene]-piperidin-1-yl}-1-cyclopropyl-6-fluoro-8-methoxy-4-oxo-1,4-dihydro-quinoline-3-carboxylic acid

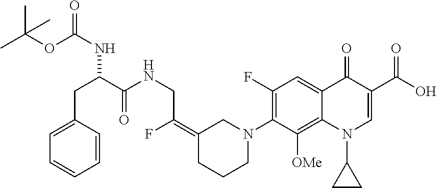

Neat ClCO₂CH₂CH₃ (1.8 mL, 18.4 mmol) was added to a THF solution (200 mL) of (S)-2-tert-butoxycarbonylamino-3-phenyl-propionic acid (4.7 g, 17.5 mmol) and i-Pr₂NEt (9.1 mL, 52.6 mmol). After 1 h, solid 7-[3-(2-amino-1-fluoro-ethylidene)-piperidin-1-yl]-1-cyclopropyl-6-fluoro-8-methoxy-4-oxo-1,4-dihydro-quinoline-3-carboxylic acid (8.0 g, 17.6 mmol) was added to the cloudy mixture. After 16 h, the resulting homogeneous solution was diluted with EtOAc and washed with water and brine, then dried (Na₂SO₄), concentrated and purified via column chromatography to yield yellow gum.

MS m/z 667 (M+H).

### STEP B: 7-{3-[2-((2S)-2-Amino-3-phenyl-propionylamino)-1-fluoro-ethylidene]-piperidin-1-yl}-1-cyclopropyl-6-fluoro-8-methoxy-4-oxo-1,4-dihydro-quinoline-3-carboxylic acid hydrochloride

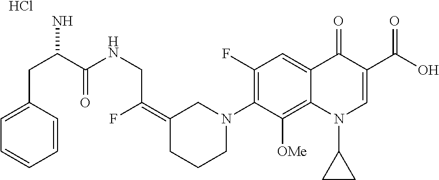

A 4 M HCl solution in dioxane (30 mL, 120 mmol) was added to a CH₂Cl₂ solution (100 mL) of 7-{3-[2-((2S)-2-tert-butoxycarbonylamino-3-phenyl-propionylamino)-1-fluoro-ethylidene]-piperidin-1-yl}-1-cyclopropyl-6-fluoro-8-methoxy-4-oxo-1,4-dihydro-quinoline-3-carboxylic acid (8.0 g, 12.0 mmol). After 2 h, the liquid was decanted off of the residue on the bottom of the flask. The residue was washed with CH₂Cl₂ (3×) and then dried in vacuo to yield the title compound as a yellow solid.

¹H NMR (300 MHz, DMSO-d₆) δ 14.96 (br. s., 1H), 8.90 (s, 1H), 8.67-8.75 (m, 1H), 8.17-8.34 (m, 3H), 7.71-7.83 (m, 1H), 7.28-7.33 (m, 1H), 7.15-7.25 (m, 2H), 4.11-4.27 (m, 2H), 4.02-4.09 (m, 3H), 3.83-4.02 (m, 2H), 3.73 (s, 3H), 3.44 (q, J=6.78 Hz, 4H), 2.98 (d, J=6.78 Hz, 2H), 2.39 (br. s., 2H), 1.73 (br. s., 2H), 0.98-1.24 (m, 2H); MS m/z 567 (M+H).

### Example 6

### Compound #15

### 1-Cyclopropyl-7-[3-(2-{[(2S)-1-[((2S)-2,6-diamino-hexanoyl)-pyrrolidine-2-carbonyl]-amino}-1-fluoro-ethylidene)-piperidin-1-yl]-6-fluoro-8-methoxy-4-oxo-1,4-dihydro-quinoline-3-carboxylic acid dihydrochloride

### STEP A: 7-(3-{2-[((2S)-1-tert-Butoxycarbonyl-pyrrolidine-2-carbonyl)-amino]-1-fluoro-ethylidene}-piperidin-1-yl)-1-cyclopropyl-6-fluoro-8-methoxy-4-oxo-1,4-dihydro-quinoline-3-carboxylic acid

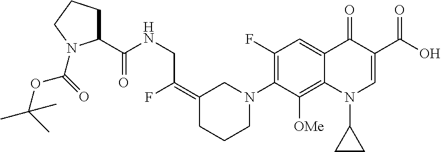

Solid HATU (6.7 g, 17.5 mmol) was added to a THF solution (200 mL) of 7-[3-(2-amino-1-fluoro-ethylidene)-piperidin-1-yl]-1-cyclopropyl-6-fluoro-8-methoxy-4-oxo-1,4-dihydro-quinoline-3-carboxylic acid hydrochloride (8.0 g, 17.5 mmol), (S)-pyrrolidine-1,2-dicarboxylic acid 1-tert-butyl ester (3.8 g, 17.5 mmol) and i-Pr₂NEt (9.7 mL, 55.5 mmol) and the resulting mixture was warmed to 40° C. After 16 h, at 40° C., the resulting mixture was diluted with EtOAc, washed with water and brine, then dried (Na₂SO₄), concentrated and purified via column chromatography to yield a yellow gum.

MS m/z 617 (M+H).

### STEP B: 1-Cyclopropyl-6-fluoro-7-(3-{1-fluoro-2-[((2S)-pyrrolidine-2-carbonyl)-amino]-ethylidene}-piperidin-1-yl)-8-methoxy-4-oxo-1,4-dihydro-quinoline-3-carboxylic acid hydrochloride

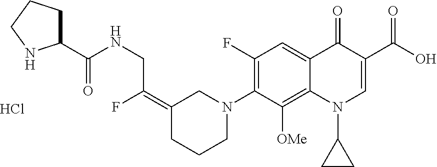

A 4 M HCl solution in dioxane (32 mL, 128 mmol) was added to a CH₂Cl₂ solution (100 mL) of 7-(3-{2-[((2S)-1-tert-butoxycarbonyl-pyrrolidine-2-carbonyl)-amino]-1-fluoro-ethylidene}-piperidin-1-yl)-1-cyclopropyl-6-fluoro-8-methoxy-4-oxo-1,4-dihydro-quinoline-3-carboxylic acid (8.0 g, 13.0 mmol). After 2 h, the liquid was decanted off of the residue on the bottom of the flask. The residue was washed with CH₂Cl₂ (3×) and then dried in vacuo to yield the title compound as a yellow solid.

MS m/z 517 (M+H).

### STEP C: 7-[3-(2-{[(2S)-1-((2S)-2,6-Bis-tert-butoxycarbonylamino-hexanoyl)-pyrrolidine-2-carbonyl]-amino}-1-fluoro-ethylidene)-piperidin-1-yl]-1-cyclopropyl-6-fluoro-8-methoxy-4-oxo-1,4-dihydro-quinoline-3-carboxylic acid

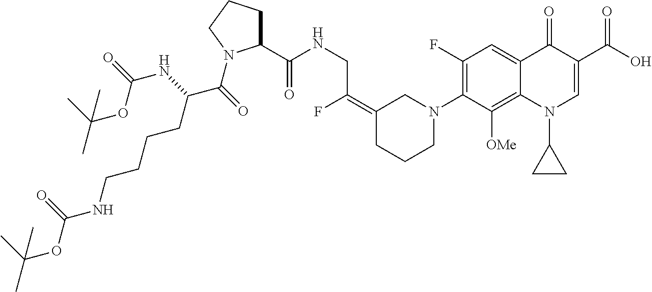

Solid HATU (4.5 g, 11.9 mmol) was added to a THF solution (200 mL) of 1-cyclopropyl-6-fluoro-7-(3-{1-fluoro-2-[((2S)-pyrrolidine-2-carbonyl)-amino]-ethylidene}-piperidin-1-yl)-8-methoxy-4-oxo-1,4-dihydro-quinoline-3-carboxylic acid hydrochloride (7.0 g, 11.9 mmol), (S)-2,6-bis-tert-butoxycarbonylamino-hexanoic acid (4.9 g, 14.3 mmol) and i-Pr₂NEt (6.1 mL, 35.6 mmol) and the resulting mixture was warmed to 40° C. After 16 h at 40° C., the resulting mixture was diluted with EtOAc, washed with water and brine, then dried (Na₂SO₄), concentrated and purified via column chromatography to yield the title compound as a yellow gum.

MS m/z 845 (M+H).

### STEP D: 1-Cyclopropyl-7-[3-(2-{[(2S)-1-((2S)-2,6-diamino-hexanoyl)-pyrrolidine-2-carbonyl]-amino}-1-fluoro-ethylidene)-piperidin-1-yl]-6-fluoro-8-methoxy-4-oxo-1,4-dihydro-quinoline-3-carboxylic acid dihydrochloride

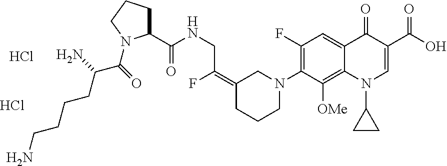

A 4 M HCl solution in dioxane (49 mL, 196 mmol) was added to a CH₂Cl₂ solution (100 mL) of 7-[3-(2-{[(2S)-2,6-Bis-tert-butoxycarbonylamino-hexanoyl)-pyrrolidine-2-carbonyl]-amino}-1-fluoro-ethylidene)-piperidin-1-yl]-1-cyclopropyl-6-fluoro-8-methoxy-4-oxo-1,4-dihydro-quinoline-3-carboxylic acid (8.2 g, 9.7 mmol). After 2 h, the liquid was decanted off of the residue on the bottom of the flask. The residue was washed with CH₂Cl₂ (3×) and then dried in vacuo to yield the title compound as a yellow solid.

¹H NMR (400 MHz, DMSO-d₆) δ 14.96 (br. s., 1H), 8.71 (s, 1H), 7.74 (d, J=11.98 Hz, 1H), 5.68 (s, 1H), 4.31 (br. s., 1H), 4.07 (br. s., 3H), 3.91 (br. s., 2H), 3.55 (s, 2H), 3.40 (br. s., 3H), 2.70-2.80 (m, 3H), 2.68 (s, 3H), 2.37 (br. s., 2H), 2.07 (br. s., 1H), 1.70 (br. s., 4H), 1.57 (br. s., 1H), 1.52 (br. s., 2H), 1.39 (br. s., 2H), 1.20-1.29 (m, 4H), 1.13 (br. s., 3H), 1.00 (br. s., 2H); MS m/z 645 (M+H).

### Example 7

### Compound #10

### 7-(3-{2-[(2S)-2-(2S)-2-Amino-propionylamino)-4-methyl-pentanoylamino]-1-fluoro-ethylidene}-piperidin-1-yl)-1-cyclopropyl-6-fluoro-8-methoxy-4-oxo-1,4-dihydro-quinoline-3-carboxylic acid hydrochloride

### STEP A: (2S)-2-((2S)-2-tert-Butoxycarbonylamino-propionylamino)-4-methyl-pentanoic acid

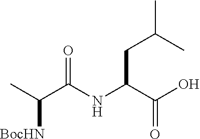

Solid (Boc)₂O was added to a THF/water solution (100 mL/20 mL) of (S,S)2-(2-amino-propionylamino)-4-methyl-pentanoic acid (5.0 g, 24.7 mmol). After 3 h, the resulting mixture was diluted with EtOAc, washed with water and brine, dried (Na₂SO₄), concentrated and the resulting residue used in the next step without further purification.

### STEP B: 7-(3-{2-[(2S)-2-((2S)-2-tert-Butoxycarbonylamino-propionylamino)-4-methyl-pentanoylamino]-1-fluoro-ethylidene}-piperidin-1-yl)-1-cyclopropyl-6-fluoro-8-methoxy-4-oxo-1,4-dihydro-quinoline-3-carboxylic acid

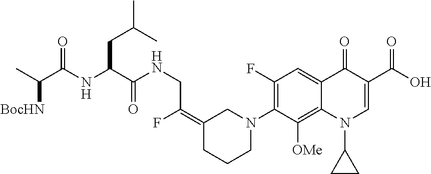

Solid HATU (7.0 g, 18.5 mmol) was added to a THF solution (200 mL) of 7-[3-(2-amino-1-fluoro-ethylidene)-piperidin-1-yl]-1-cyclopropyl-6-fluoro-8-methoxy-4-oxo-1,4-dihydro-quinoline-3-carboxylic acid hydrochloride (8.4 g, 18.5 mmol), (S,S)-2-(2-tert-butoxycarbonylamino-propionylamino)-4-methyl-pentanoic acid (5.6 g, 18.5 mmol) and i-Pr₂NEt (9.7 mL, 55.5 mmol) and the resulting mixture was warmed to 40° C. After 16 h at 40° C., the resulting mixture was diluted with EtOAc, washed with water and brine, then dried (Na₂SO₄), concentrated and purified via column chromatography to yield the title compound as a yellow gum.

¹H NMR (CHLOROFORM-d, 300 MHz): δ=14.88 (br. s., 1H), 8.80 (s, 1H), 7.67-7.97 (m, 1H), 6.53-6.70 (m, 1H), 5.31 (s, 1H), 5.01-5.19 (m, 1H), 4.35-4.50 (m, 1H), 4.16 (br. s., 1H), 4.02-4.13 (m, 3H), 3.99 (s, 2H), 3.77 (s, 3H), 3.43-3.50 (m, 2H), 2.40-2.50 (m, 2H), 1.73-1.86 (m, 2H), 1.48-1.72 (m, 3H), 1.43 (d, J=2.3 Hz, 9H), 1.35 (d, J=7.2 Hz, 3H), 1.21-1.31 (m, 2H), 0.99-1.08 (m, 2H), 0.87 (d, J=6.0 Hz, 3H), 0.90 ppm (d, J=6.4 Hz, 3H).

### STEP C: 7-(3-{2-[(2S)-2-((2S)-2-Amino-propionylamino)-4-methyl-pentanoylamino]-1-fluoro-ethylidene}-piperidin-1-yl)-1-cyclopropyl-6-fluoro-8-methoxy-4-oxo-1,4-dihydro-quinoline-3-carboxylic acid hydrochloride

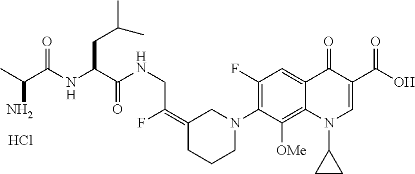

A 4 M HCl solution in dioxane (70 mL, 284.4 mmol) was added to a CH₂Cl₂ solution (70 mL) of 7-(3-{2-[(2S)-2-((2S)-2-tert-Butoxycarbonylamino-propionylamino)-4-methyl-pentanoylamino]-1-fluoro-ethylidene}-piperidin-1-yl)-1-cyclopropyl-6-fluoro-8-methoxy-4-oxo-1,4-dihydro-quinoline-3-carboxylic acid (10.0 g, 14.2 mmol). After 2 h, the liquid was decanted off of the residue on the bottom of the flask. The residue was washed with CH₂Cl₂ (3×) and then dried in vacuo to yield the title compound as a yellow solid.

¹H NMR (DMSO-d₆, 300 MHz): δ=8.71 (s, 1H), 8.38-8.58 (m, 2H), 8.04-8.26 (m, 3H), 7.75 (d, J=12.4 Hz, 1H), 4.31 (td, J=8.8, 5.5 Hz, 1H), 4.13-4.23 (m, 1 H), 4.01 (d, J=5.3 Hz, 1H), 3.96 (br. s., 2H), 3.81 (d, J=11.3 Hz, 1H), 3.74 (s, 3H), 3.32-3.47 (m, 3H), 2.51 (d, J=1.5 Hz, 2H), 2.38 (br. s., 2H), 1.65-1.81 (m, 2H), 1.49-1.64 (m, 1H), 1.30-1.48 (m, 2H), 1.27 (d, J=6.8 Hz, 3H), 0.97-1.19 (m, 2H), 0.76-0.91 ppm (m, 6H); MS m/z 604 (M+H).

### Example 8

### Compound #16

### 7-(3-{2-[2-((2S)-2-Amino-4-carboxy-butyrylamino)-acetylamino]-1-fluoro-ethylidene}-piperidin-1-yl)-1-cyclopropyl-6-fluoro-8-methoxy-4-oxo-1,4-dihydro-Quinoline-3-carboxylic acid hydrochloride

### STEP A: 7-(3-{2-[2-((2S)-4-tert-Butoxycarbonyl-2-tert-butoxycarbonylamino-butyrylamino)-acetylamino]-1-fluoro-ethylidene}-piperidin-1-yl)-1-cyclopropyl-6-fluoro-8-methoxy-4-oxo-1,4-dihydro-quinoline-3-carboxylic acid

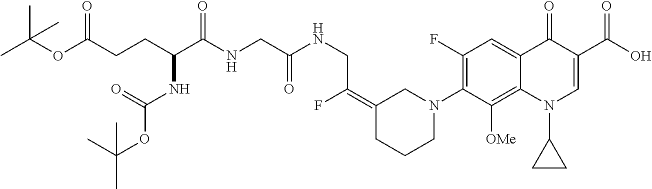

Solid HATU (5.4 g, 14.3 mmol) was added to a THF solution (130 mL) of 7-{3-[2-(2-amino-acetylamino)-1-fluoro-ethylidene]-piperidin-1-yl}-1-cyclopropyl-6-fluoro-8-methoxy-4-oxo-1,4-dihydro-quinoline-3-carboxylic acid hydrochloride (7.0 g, 13.6 mmol) (prepared according to the procedure described in Example 2 above), (S)-2-tert-butoxycarbonylamino-pentanedioic acid 5-tert-butyl ester (4.3 g, 14.3 mmol) and 1-Pr₂NEt (11.9 mL, 68.2 mmol) and the resulting mixture was warmed to 40° C. After 16 h at 40° C., the resulting mixture was diluted with EtOAc, washed with water and brine, dried (Na₂SO₄), concentrated and purified via column chromatography to yield a yellow gum.

¹H NMR (CHLOROFORM-d, 400 MHz): δ=14.85 (s, 1H), 8.82 (s, 1H), 7.86 (d, J=12.2 Hz, 1H), 6.95-7.13 (m, 1H), 6.70-6.87 (m, 1H), 5.52 (br. s., 1H), 4.21 (br. s., 1H), 4.16-4.26 (m, 1H), 3.85-4.09 (m, 6H), 3.77 (s, 3H), 3.46 (br. s., 2H), 2.39-2.49 (m, 3H), 2.28-2.38 (m, 1H), 2.01-2.11 (m, 1H), 1.87-2.00 (m, 1H), 1.79 (br. s., 2H), 1.45 (s, 9H), 1.43 (s, 9H), 1.23-1.30 (m, 2H), 0.98-1.08 ppm (m, 2H).

### STEP B: 7-(3-{2-[2-((2S)-2-Amino-4-carboxy-butyrylamino)-acetylamino]-1-fluoro-ethylidene}-piperidin-1-yl)-1-cyclopropyl-6-fluoro-8-methoxy-4-oxo-1,4-dihydro-quinoline-3-carboxylic acid hydrochloride

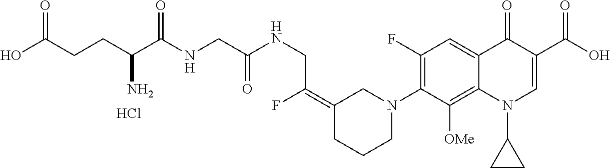

A 4 M HCl solution in dioxane (59 mL, 236.3 mmol) was added to a CH₂Cl₂ solution (60 mL) of 7-(3-{2-[2-((2S)-4-tert-butoxycarbonyl-2-tert-butoxycarbonylamino-butyrylamino)-acetylamino]-1-fluoro-ethylidene}-piperidin-1-yl)-1-cyclopropyl-6-fluoro-8-methoxy-4-oxo-1,4-dihydro-quinoline-3-carboxylic acid (9.0 g, 11.8 mmol). After 2 h, the liquid was decanted off of the residue on the bottom of the flask. The residue was washed with CH₂Cl₂ (3×) and then dried in vacuo to yield the title compound as a yellow solid.

¹H NMR (DMSO-d₆, 400 MHz): δ=8.84 (t, J=5.9 Hz, 1H), 8.70 (s, 1H), 8.48 (t, J=5.4 Hz, 1H), 8.41 (br. s., 3H), 7.76 (d, J=12.2 Hz, 1H), 4.13-4.23 (m, 1H), 3.90-4.13 (m, 4H), 3.79-3.88 (m, 1H), 3.75-3.78 (m, 1H), 3.74 (s, 3H), 3.42 (br. s., 2H), 2.32-2.41 (m, 4H), 1.85-2.03 (m, 2H), 1.67-1.77 (m, 2H), 1.60 (s, 1H), 1.14 (d, J=6.8 Hz, 2H), 0.99-1.06 ppm (m, 2H); MS m/z 606 (M+H).

### Example 9

### Compound #14

### 7-{3-[2-((2S)-2-Amino-3-dimethylamino-propionylamino)-1-fluoro-ethylidene]-piperidin-1-yl}-1-cyclopropyl-6-fluoro-8-methoxy-4-oxo-1,4-dihydro-quinoline-3-carboxylic acid dihydrochloride

### STEP A: 7-{3-[2-((2S)-2-tert-Butoxycarbonylamino-3-dimethylamino-propionylamino)-1-fluoro-ethylidene]-piperidin-1-yl}-1-cyclopropyl-6-fluoro-8-methoxy-4-oxo-1,4-dihydro-quinoline-3-carboxylic acid

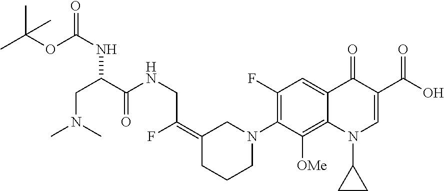

Solid HATU (7.9 g, 20.7 mmol) was added to a THF solution (200 mL) of 7-[3-(2-amino-1-fluoro-ethylidene)-piperidin-1-yl]-1-cyclopropyl-6-fluoro-8-methoxy-4-oxo-1,4-dihydro-quinoline-3-carboxylic acid (9.0 g, 19.7 mmol), (S)-2-tert-butoxycarbonylamino-3-dimethylamino-propionic acid (4.8 g, 20.7 mmol) and i-Pr₂NEt (10.3 mL, 59.2 mmol) and the resulting mixture was warmed to 45° C. After 16 h at 45° C., the resulting mixture was diluted with EtOAc, washed with water and brine, then dried (Na₂SO₄), concentrated and purified via column chromatography to yield a yellow gum.

¹H NMR (CHLOROFORM-d, 400 MHz): δ=8.75-8.86 (m, 1H), 8.45 (br. s., 1H), 7.85 (d, J=12.2 Hz, 1H), 5.51 (br. s., 1H), 4.13-4.30 (m, 1H), 4.03-4.12 (m, 3 H), 4.00 (br. s., 2H), 3.77 (s, 3H), 3.68-3.75 (m, 1H), 3.41-3.51 (m, 2H), 3.21 (q, J=7.3 Hz, 1H), 2.81 (s, 6H), 2.62-2.73 (m, 1H), 2.57 (d, J=10.3 Hz, 1H), 1.73-1.88 (m, 2H), 1.43 (s, 9H), 1.22-1.33 (m, 2H), 0.95-1.09 ppm (m, 2H); MS m/z 634 (M+H).

### STEP B: 7-{3-[2-((2S)-2-Amino-3-dimethylamino-propionylamino)-1-fluoro-ethylidene]-piperidin-1-yl}-1-cyclopropyl-6-fluoro-8-methoxy-4-oxo-1,4-dihydro-quinoline-3-carboxylic acid dihydrochloride

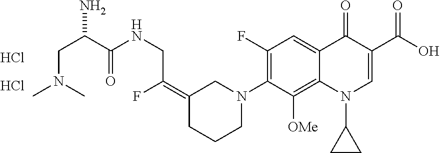

A 4 M HCl solution in dioxane (67 mL, 268.3 mmol) was added to a CH₂Cl₂ solution (64 mL) of 7-{3-[2-((2S)-2-tert-butoxycarbonylamino-3-dimethylamino-propionylamino)-1-fluoro-ethylidene]-piperidin-1-yl}-1-cyclopropyl-6-fluoro-8-methoxy-4-oxo-1,4-dihydro-quinoline-3-carboxylic acid (8.5 g, 13.4 mmol). After 2 h, the liquid was decanted off of the residue on the bottom of the flask. The residue was washed with CH₂Cl₂ (3×) and then dried in vacuo to yield the title compound as a yellow solid.

¹H NMR (DMSO-d₆, 400 MHz): δ=10.97 (br. s., 1H), 9.50 (t, J=5.4 Hz, 1H), 8.80 (br. s., 3H), 8.71 (s, 1H), 7.77 (d, J=12.2 Hz, 1H), 4.50 (br. s., 1H), 4.04-4.27 (m, 3H), 3.89-4.04 (m, 2H), 3.75 (s, 3H), 3.38-3.52 (m, 4H), 2.87 (br. s., 6H), 2.40 (dt, J=12.3, 6.4 Hz, 2H), 1.74 (br. s., 2H), 1.28 (dd, J=13.2, 6.6 Hz, 1H), 1.10-1.20 (m, 2H), 0.99-1.09 ppm (m, 2H); MS m/z 534 (M+H).

### Example 10

### Compound #4

### 7-{3-[2-((2S)-2-Amino-propionylamino)-1-fluoro-ethylidene]-piperidin-1-yl}-1-cyclopropyl-6-fluoro-8-methoxy-4-oxo-1,4-dihydro-quinoline-3-carboxylic acid hydrochloride

### STEP A: 7-{3-[2-((2S)-2-tert-Butoxycarbonylamino-propionylamino)-1-fluoro-ethylidene]-piperidin-1-yl}-1-cyclopropyl-6-fluoro-8-methoxy-4-oxo-1,4-dihydro-quinoline-3-carboxylic acid

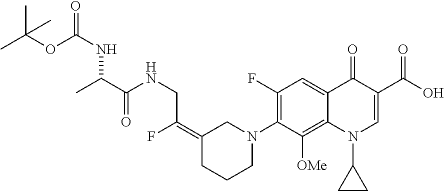

Solid HATU (7.3 g, 19.3 mmol) was added to a THF solution (200 mL) of 7-[3-(2-amino-1-fluoro-ethylidene)-piperidin-1-yl]-1-cyclopropyl-6-fluoro-8-methoxy-4-oxo-1,4-dihydro-quinoline-3-carboxylic acid (8.0 g, 17.5 mmol), L-Boc-alanine (3.7 g, 19.3 mmol) and Et₃N (6.1 mL, 43.7 mmol). After 4 h at room temperature, the resulting mixture was diluted with EtOAc, washed with saturated aqueous NaHCO₃ and brine, then dried (Na₂SO₄), concentrated and purified via column chromatography to yield a white solid.

MS m/z 591 (M+H).

### STEP B: 7-{3-[2-((2S)-2-Amino-propionylamino)-1-fluoro-ethylidene]-piperidin-1-yl}-1-cyclopropyl-6-fluoro-8-methoxy-4-oxo-1,4-dihydro-quinoline-3-carboxylic acid hydrochloride

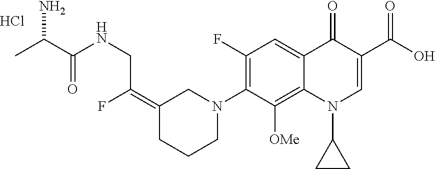

A 4 M HCl solution in dioxane (60 mL, 240.0 mmol) was added to a CH₂Cl₂ solution (60 mL) of 7-{3-[2-((2S)-2-tert-butoxycarbonylamino-propionylamino)-1-fluoro-ethylidene]-piperidin-1-yl}-1-cyclopropyl-6-fluoro-8-methoxy-4-oxo-1,4-dihydro-quinoline-3-carboxylic acid (6.6 g, 17.5 mmol). After 2 h, the liquid was decanted off of the residue on the bottom of the flask. The residue was washed with CH₂Cl₂ (3×) and then dried in vacuo to yield the title compound as a yellow solid.

¹H NMR (400 MHz, DMSO-d₆) δ 8.85 (t, J=5.50 Hz, 1H), 8.71 (s, 1H), 8.16 (bs, 3H), 7.77 (d, J=11.98 Hz, 1H), 4.03-4.22 (m, 3H), 3.90-4.03 (m, 2H), 3.65-3.87 (m, 4H), 3.43 (br. s., 1H), 2.69 (s, 1H), 1.73 (br. s., 2H), 2.55-2.30 (m, 2H), 1.31 (d, J=7.09 Hz, 3H), 1.09-1.19 (m, 2H), 0.99-1.09 ppm (m, 2H); MS m/z 491 (M+H).

### Example 11

### Compound #5

### 7-{3-[2-((2S)-2-Amino-3-carboxy-propionylamino)-1-fluoro-ethylidene]-piperidin-1-yl}-1-cyclopropyl-6-fluoro-8-methoxy-4-oxo-1,4-dihydro-quinoline-3-carboxylic acid hydrochloride

### STEP A: 7-{3-[2-((2S)-3-tert-Butoxycarbonyl-2-tert-butoxycarbonylamino-propionylamino)-1-fluoro-ethylidene]-piperidin-1-yl}-1-cyclopropyl-6-fluoro-8-methoxy-4-oxo-1,4-dihydro-quinoline-3-carboxylic acid

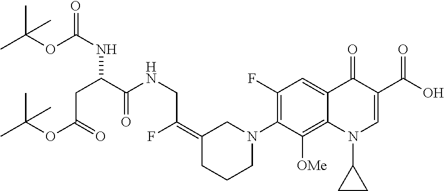

Solid HATU (7.3 g, 19.3 mmol) was added to a THF solution (200 mL) of 7-[3-(2-amino-1-fluoro-ethylidene)-piperidin-1-yl]-1-cyclopropyl-6-fluoro-8-methoxy-4-oxo-1,4-dihydro-quinoline-3-carboxylic acid (8.0 g, 17.5 mmol), (S)-2-tert-butoxycarbonylamino-succinic acid 4-tert-butyl ester (5.6 g, 19.3 mmol) and Et₃N (6.1 mL, 43.7 mmol). After 4 h at room temperature, the resulting mixture was diluted with EtOAc, washed with saturated aqueous NaHCO₃ and brine, then dried (Na₂SO₄), concentrated and purified via column chromatography to a white solid.

MS m/z 691 (M+H).

### STEP B: 7-{3-[2-((2S)-2-Amino-3-carboxy-propionylamino)-1-fluoro-ethylidene]-piperidin-1-yl}-1-cyclopropyl-6-fluoro-8-methoxy-4-oxo-1,4-dihydro-quinoline-3-carboxylic acid hydrochloride

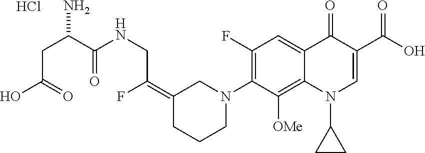

Neat TFA (120 mL, 1.6 mol) was added to a CH₂Cl₂ solution (120 mL) of 7-{3-[2-((2S)-3-tert-butoxycarbonyl-2-tert-butoxycarbonylamino-propionylamino)-1-fluoro-ethylidene]-piperidin-1-yl}-1-cyclopropyl-6-fluoro-8-methoxy-4-oxo-1,4-dihydro-quinoline-3-carboxylic acid (11.0 g, 15.9 mmol). After 4 h, the resulting mixture was concentrated, dissolved in THF and 1 N HCl in ethyl ether (50 mL) was added. The resulting precipitate was filtered and dried in vacuo. F¹⁹NMR indicated that the TFA salt was not completely converted to the HCl salt. The solid was suspended in dichloromethane (100 mL) and 1N HCl/ethyl ether (50 mL) and stirred for 3 h at room temperature. The resulting solid was filtered and dried in vacuo to yield the title compound as a yellow solid.

¹H NMR (400 MHz, DMSO-d₆) δ 8.88 (t, J=5.26 Hz, 1H), 8.71 (s, 1H), 8.23 (br. s., 3H), 7.77 (d, J=12.23 Hz, 1H), 3.90-4.22 (m, 5H), 3.74 (s, 3H), 3.45 (br. s., 2H), 2.72-2.89 (m, 2H), 2.69 (s, 1H), 2.30-2.45 (m, 2H), 1.68-1.77 (m, 2H), 1.09-1.18 (m, 2H), 1.00-1.09 ppm (m, 2H); MS m/z 535 (M+H).

### Example 12

### Compound #7

### 1-Cyclopropyl-6-fluoro-7-{3-[1-fluoro-2-((2S)-2-methylamino-propionylamino]-ethylidene}-piperidin-1-yl)-8-methoxy-4-oxo-1,4-dihydro-quinoline-3-carboxylic acid hydrochloride

### STEP A: 7-(3-{2-[(2S)-2-(tert-Butoxycarbonyl-methyl-amino)-propionylamino]-1-fluoro-ethylidene}-piperidin-1-yl)-1-cyclopropyl-6-fluoro-8-methoxy-4-oxo-1,4-dihydro-quinoline-3-carboxylic acid

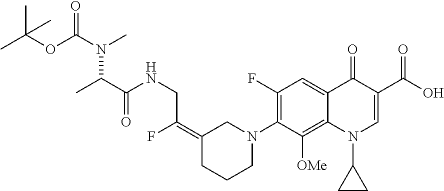

Solid HATU (7.3 g, 19.3 mmol) was added to a THF solution (200 mL) of 7-[3-(2-amino-1-fluoro-ethylidene)-piperidin-1-yl]-1-cyclopropyl-6-fluoro-8-methoxy-4-oxo-1,4-dihydro-quinoline-3-carboxylic acid (8.0 g, 17.5 mmol), (S)-2-(tert-butoxycarbonyl-methyl-amino)-propionic acid (3.9 g, 19.3 mmol) and Et₃N (6.1 mL, 43.7 mmol). After 4 h at room temperature, the resulting mixture was diluted with EtOAc, washed with saturated aqueous NaHCO₃ and brine, then dried (Na₂SO₄), concentrated and purified via column chromatography to yield a white solid.

MS m/z 605 (M+H).

### STEP B: 1-Cyclopropyl-6-fluoro-7-{3-[1-fluoro-2-((2S)-2-methylamino-propionylamino)-ethylidene}-piperidin-1-yl]-8-methoxy-4-oxo-1,4-dihydro-quinoline-3-carboxylic acid hydrochloride

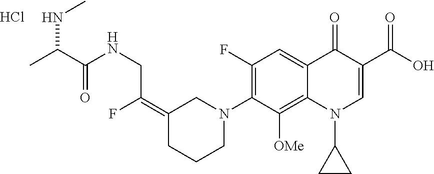

Neat TFA (80 mL, 1.1 mol) was added to a CH₂Cl₂ solution (100 mL) of 7-(3-{2-[(2S)-2-(tert-butoxycarbonyl-methyl-amino)-propionylamino]-1-fluoro-ethylidene}-piperidin-1-yl)-1-cyclopropyl-6-fluoro-8-methoxy-4-oxo-1,4-dihydro-quinoline-3-carboxylic acid (10.0 g, 16.5 mmol). After 4 h, the resulting mixture was concentrated, dissolved in CH₂Cl₂, 1 N HCl in ethyl ether (50 mL) was added and the resulting mixture was concentrated. This procedure was repeated twice more and the resulting solid was dried in vacuo to yield the title compound as a white solid.

¹H NMR (400 MHz, DMSO-d₆) δ 9.20 (br. s., 1H), 8.99 (t, J=5.50 Hz, 1H), 8.80 (br. s., 1H), 8.71 (s, 1H), 7.77 (d, J=11.98 Hz, 1H), 4.07-4.21 (m, 3H), 3.97 (s, 2H), 3.71-3.79 (m, 4H), 3.39-3.46 (m, 2H), 2.32-2.45 (m, 5H), 1.73 (br. s., 2H), 1.34 (d, J=6.85 Hz, 3H), 1.02-1.18 ppm (m, 4H); MS m/z 505 (M+H⁺).

### Example 13

### Compound #8

### 1-Cyclopropyl-6-fluoro-7-{3-[1-fluoro-2-(5-methyl-2-oxo-[1,3]-dioxol-4-ylmethoxycarbonylamino)-ethylidene]-piperidin-1-yl}-8-methoxy-4-oxo-1,4-dihydro-quinoline-3-carboxylic acid

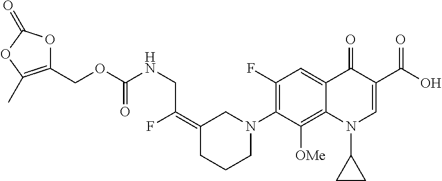

Solid carbonic acid 5-methyl-2-oxo-[1,3]dioxol-4-ylmethyl ester 4-nitro-phenyl ester (5.4 g, 18.5 mmol) was added to a THF solution (160 mL) of 7-[3-(2-amino-1-fluoro-ethylidene)-piperidin-1-yl]-1-cyclopropyl-6-fluoro-8-methoxy-4-oxo-1,4-dihydro-quinoline-3-carboxylic acid (8.0 g, 17.6 mmol) and i-Pr₂NEt (6.1 mL, 35.2 mmol). After 6 h, the resulting mixture was diluted with EtOAc, washed with water and brine, then dried (Na₂SO₄), concentrated and purified via column chromatography to yield the title compound as a yellow solid.

¹H NMR (CHLOROFORM-d, 300 MHz): δ=14.77 (s, 1H), 8.81 (s, 1H), 7.85 (d, J=12.1 Hz, 1H), 5.26 (t, J=5.7 Hz, 1H), 4.77 (s, 2H), 4.01-4.20 (m, 1H), 3.97 (s, 2H), 3.78 (s, 3H), 3.37-3.56 (m, 2H), 2.47 (t, J=5.5 Hz, 2H), 2.13 (s, 3H), 1.72-1.91 (m, 2H), 1.64 (s, 2H), 1.16-1.36 (m, 2H), 0.92-1.11 ppm (m, 2H); MS m/z 576 (M+H).

### Example 14

### Compound #13

### 1-Cyclopropyl-7-{3-[2-(diethoxy-phosphorylamino)-1-fluoro-ethylidene]-piperidin-1-yl}-6-fluoro-8-methoxy-4-oxo-1,4-dihydro-quinoline-3-carboxylic acid sodium salt

### STEP A: 1-Cyclopropyl-7-{3-[2-(diethoxy-phosphorylamino)-1-fluoro-ethylidene]-piperidin-1-yl}-6-fluoro-8-methoxy-4-oxo-1,4-dihydro-quinoline-3-carboxylic acid

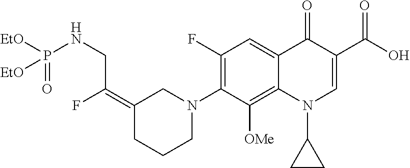

Neat (CH₃CH₂O)₂POCl (3.5 mL, 24.2 mmol) was added to a THF solution (300 mL) of 7-[3-(2-amino-1-fluoro-ethylidene)-piperidin-1-yl]-1-cyclopropyl-6-fluoro-8-methoxy-4-oxo-1,4-dihydro-quinoline-3-carboxylic acid (10.0 g, 22.0 mmol) and 1-Pr₂NEt (11.5 mL, 66.0 mmol). After 4 h, the resulting mixture was diluted with EtOAc, washed with water and brine, then dried (Na₂SO₄), concentrated and purified via column chromatography to yield a yellow solid.

¹H NMR (CHLOROFORM-d, 300 MHz): δ=14.74 (s, 1H), 8.82 (s, 1H), 7.88 (d, J=12.1 Hz, 1H), 3.96-4.16 (m, 4H), 3.91 (s, 2H), 3.83 (dd, J=10.5, 6.8 Hz, 1H), 3.78 (s, 3H), 3.74 (d, J=7.2 Hz, 1H), 3.40-3.51 (m, 2H), 2.87-3.03 (m, 1H), 2.47 (t, J=5.7 Hz, 2H), 1.73-1.86 (m, 2H), 1.20-1.34 (m, 6H), 0.96-1.07 ppm (m, 2H);

MS m/z 556 (M+H).

### STEP B: 1-Cyclopropyl-7-{3-[2-(diethoxy-phosphorylamino)-1-fluoro-ethylidene]-piperidin-1-yl}-6-fluoro-8-methoxy-4-oxo-1,4-dihydro-quinoline-3-carboxylic acid sodium salt

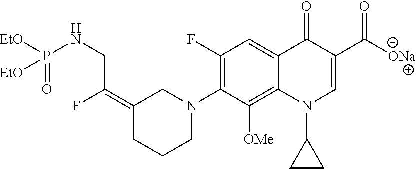

A 1 M aqueous NaOH solution (17.1 mL, 17.1 mmol) was added to a CH₃CN solution of 1-cyclopropyl-7-{3-[2-(diethoxy-phosphorylamino)-1-fluoro-ethylidene]-piperidin-1-yl}-6-fluoro-8-methoxy-4-oxo-1,4-dihydro-quinoline-3-carboxylic acid (9.5 g, 17.1 mmol). After 1 h, the resulting mixture was concentrated in vacuo, washed with water (2×), and dried in vacuo to yield the title compound as a yellow solid.

¹H NMR (DMSO-d₆, 300 MHz): δ=8.57 (s, 1H), 7.64 (d, J=12.8 Hz, 1H), 5.27-5.39 (m, 1H), 3.98 (dt, J=7.0, 3.3 Hz, 1H), 3.83 (quin, J=7.3 Hz, 6H), 3.71 (s, 3H), 3.51-3.69 (m, 2H), 2.36 (br. s., 2H), 1.70 (br. s., 2H), 1.15 (t, J=7.2 Hz, 6H), 1.03-1.11 (m, 1H), 0.86 ppm (br. s., 2H); MS m/z 556 (M+H).

### Example 15

### Compound #11

### 7-[3-(2-Amino-1-fluoro-ethylidene)-piperidin-1-yl]-1-cyclopropyl-6-fluoro-8-methoxy-4-oxo-1,4-dihydro-quinoline-3-carboxylic acid ethyl ester hydrochloride

### STEP A: 7-[3-(2-tert-Butoxycarbonylamino-1-fluoro-ethylidene)-piperidin-1-yl]-1-cyclopropyl-6-fluoro-8-methoxy-4-oxo-1,4-dihydro-quinoline-3-carboxylic acid

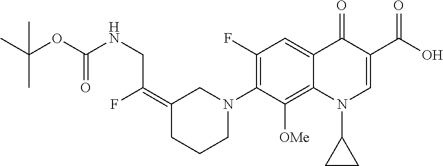

Solid Boc₂O (4 g, 18.4 mmol) was added to a CH₂Cl₂ solution (300 mL) of 7-[3-(2-amino-1-fluoro-ethylidene)-piperidin-1-yl]-1-cyclopropyl-6-fluoro-8-methoxy-4-oxo-1,4-dihydro-quinoline-3-carboxylic acid (8.0 g, 17.5 mmol) and Et₃N (6.1 mL, 43.7 mmol). The resulting mixture was stirred at room temperature for 6 h and concentrated. The resulting residue was purified by column chromatography to yield a white solid.

MS m/z 520 (M+H).

### STEP B: 7-[3-(2-tert-Butoxycarbonylamino-1-fluoro-ethylidene)-piperidin-1-yl]-1-cyclopropyl-6-fluoro-8-methoxy-4-oxo-1,4-dihydro-quinoline-3-carboxylic acid ethyl ester

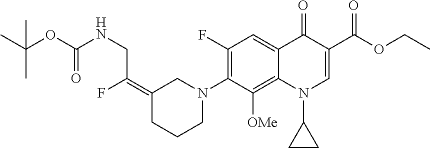

Neat CH₃CH₂I (3.6 g, 23.1 mmol) was added to a MeCN solution (300 mL) of 7-[3-(2-tert-butoxycarbonylamino-1-fluoro-ethylidene)-piperidin-1-yl]-1-cyclopropyl-6-fluoro-8-methoxy-4-oxo-1,4-dihydro-quinoline-3-carboxylic acid (10 g, 19.3 mmol) and Cs₂CO₃ (12.6 g, 38.6 mmol) and the resulting mixture was heated to 85° C. After 4 h, the resulting mixture was cooled and then concentrated. The resulting residue was treated with dichloromethane (200 mL) and filtered. The filtrate was concentrated and purified by column chromatography to yield a white solid.

MS m/z 548 (M+H).

### STEP C: 7-[3-(2-Amino-1-fluoro-ethylidene)-piperidin-1-yl]-1-cyclopropyl-6-fluoro-8-methoxy-4-oxo-1,4-dihydro-quinoline-3-carboxylic acid ethyl ester hydrochloride

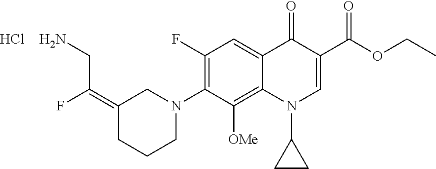

A 4 M HCl solution in dioxane (80 mL, 320 mmol) was added to a CH₂Cl₂ solution (80 mL) of 7-[3-(2-tert-butoxycarbonylamino-1-fluoro-ethylidene)-piperidin-1-yl]-1-cyclopropyl-6-fluoro-8-methoxy-4-oxo-1,4-dihydro-quinoline-3-carboxylic acid ethyl ester (7.6 g, 13.9 mmol). After 2 h, the liquid was decanted off of the residue on the bottom of the flask. The residue was washed with CH₂Cl₂ (3×) and then dried in vacuo to yield the title compound as a yellow solid.

¹H NMR (400 MHz, DMSO-d₆) δ 8.48 (s, 1H), 8.37 (br. s., 3H), 7.62 (d, J=12.23 Hz, 1H), 4.21 (q, J=7.09 Hz, 2H), 4.02 (dt, J=3.33, 7.27 Hz, 1H), 3.78-3.95 (m, 4H), 3.73 (s, 3H), 3.37 (br. s., 2H), 2.43 (br. s., 2H), 1.73 (br. s., 2H), 1.27 (t, J=7.09 Hz, 3H), 1.03-1.18 (m, 2H), 0.91-1.03 (m, 2H); MS (ES) m/z: 448 (M+H⁺).

### Example 16

### Compound #12

### 7-[3-(2-Amino-1-fluoro-ethylidene)-piperidin-1-yl]-1-cyclopropyl-6-fluoro-8-methoxy-4-oxo-1,4-dihydro-quinoline-3-carboxylic acid isopropyl ester hydrochloride

### STEP A: 7-[3-(2-tert-Butoxycarbonylamino-1-fluoro-ethylidene)-piperidin-1-yl]-1-cyclopropyl-6-fluoro-8-methoxy-4-oxo-1,4-dihydro-quinoline-3-carboxylic acid isopropyl ester

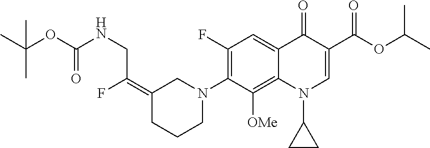

Neat i-PrI (2.8 mL, 28.1 mmol) was added to a NMP solution (100 mL) of 7-[3-(2-tert-butoxycarbonylamino-1-fluoro-ethylidene)-piperidin-1-yl]-1-cyclopropyl-6-fluoro-8-methoxy-4-oxo-1,4-dihydro-quinoline-3-carboxylic acid (11.2 g, 21.6 mmol) (prepared as described in Example 15 above) and Cs₂CO₃ (7.7 g, 23.8 mmol) and the resulting mixture was warmed to 80° C. After 5 h, the resulting mixture was cooled, diluted with EtOAc, washed with water and brine, then dried (Na₂SO₄), concentrated and purified via column chromatography to yield a yellow gum.

¹H NMR (CHLOROFORM-d, 300 MHz): δ=8.54 (s, 1H), 7.88 (d, J=12.4 Hz, 1H), 5.24 (quin, J=6.2 Hz, 1H), 4.90 (br. s., 1H), 3.97-4.10 (m, 2H), 3.83-3.96 (m, 2H), 3.75 (s, 3H), 3.35-3.47 (m, 2H), 2.43 (t, J=5.5 Hz, 2H), 1.40 (s, 9H), 1.39 (s, 3 H), 1.37 (s, 3H), 1.19 (q, J=7.0 Hz, 2H), 0.92-0.98 ppm (m, 2H).

### STEP B: 7-[3-(2-Amino-1-fluoro-ethylidene)-piperidin-1-yl]-1-cyclopropyl-6-fluoro-8-methoxy-4-oxo-1,4-dihydro-quinoline-3-carboxylic acid isopropyl ester hydrochloride

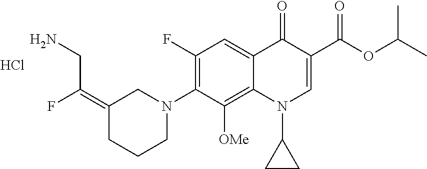

A 4 M HCl solution in dioxane (60 mL, 235.3 mmol) was added to a CH₂Cl₂ solution (60 mL) of 7-[3-(2-tert-butoxycarbonylamino-1-fluoro-ethylidene)-piperidin-1-yl]-1-cyclopropyl-6-fluoro-8-methoxy-4-oxo-1,4-dihydro-quinoline-3-carboxylic acid isopropyl ester (6.6 g, 11.8 mmol). After 2 h, the liquid was decanted off of the residue on the bottom of the flask. The residue was washed with CH₂Cl₂ (3×) and then dried in vacuo to yield the title compound as a yellow solid.

¹H NMR (DMSO-d₆, 300 MHz): δ=8.47 (br. s., 1H), 8.45 (s, 1H), 7.62 (d, J=12.4 Hz, 1H), 6.52 (br. s., 2H), 5.04 (quin, J=6.3 Hz, 1H), 3.96-4.10 (m, 1H), 3.87 (br. s., 3H), 3.76-3.83 (m, 1H), 3.73 (s, 3H), 3.37 (br. s., 2H), 2.42 (br. s., 2H), 1.73 (br. s., 2H), 1.27 (d, J=6.0 Hz, 6H), 1.06-1.16 (m, 2H), 0.89-1.00 ppm (m, 2 H); MS m/z 462 (M+H).

### Example 17

### Compound #17

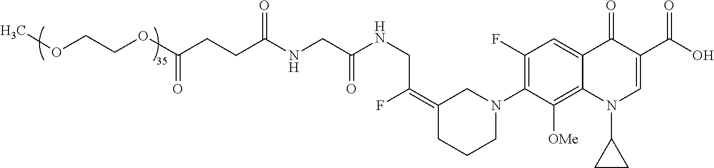

Solid mPEG-succinyl-NHS (34.9 g, 15.0 mmol, purchased from NOF Corporation, One North Broadway, Suite 1012, White Plains, N.Y. 10601) was added to a DMF solution (60 mL) of 7-{3-[2-(2-amino-acetylamino)-1-fluoro-ethylidene]-piperidin-1-yl}-1-cyclopropyl-6-fluoro-8-methoxy-4-oxo-1,4-dihydro-quinoline-3-carboxylic acid hydrochloride (7.0 g, 13.6 mmol) (prepared according to the procedure described in Example 2 above) and i-Pr₂NEt (11.9 mL, 68.2 mmol). After 16 h, the resulting mixture was concentrated and purified via HPLC to yield the title compound as a yellow solid.

¹H NMR (methanol-d₄, 400 MHz): δ=8.90 (s, 1H), 7.84 (d, J=12.5 Hz, 1H), 4.18-4.31 (m, 4H), 4.15 (s, 1H), 4.09 (s, 2H), 3.81-3.84 (m, 3H), 3.50-3.72 (m, 232H), 3.47 (dd, J=5.5, 3.8 Hz, 2H), 3.26-3.35 (m, 2H), 2.65-2.71 (m, 2H), 2.54 (t, J=6.6 Hz, 2H), 2.50 (d, J=5.4 Hz, 2H), 1.83 (br. s., 2H), 1.28 (d, J=6.8 Hz, 2H), 1.04-1.13 ppm (m, 2H)

### Biological Examples

### Example 18

### Aqueous Solubility

### Preparation of Zwitterions:

A 50-mg/mL solution of a compound of formula (I) or a compound of formula (II) in H₂O was titrated with 0.1 M NaOH until pH reached 5.7-6.1. Any solid that precipitated was filtered. The remaining solutions or phase-separated oil/H₂O mixtures were freeze-dried to produce zwitterions as fluffy solids.

### Chromatographic Conditions Used to Characterize Zwitterions:

Column. Supelco Ascentis C18, 4.6×250 mm (5 nm) Temperature 50° C.

Injection volume: 10 μL

Flow rate: 1.30 mL/min

Detection: UV at 298 nm

Mobile Phases: A=0.1% TFA/H₂O

B=0.1% TFA in 47.5:47.5:5 MeOH/ACN/H₂O

Method:

| Time (min) | % A | % B |
|---|---|---|
| 0 | 94 | 6 |
| 6 | 94 | 6 |
| 30 | 50 | 50 |
| 44 | 0 | 100 |

Procedure to Determine Clarity of Test Compound Solutions at 3.3 mg/ml (Equivalent to Active Form Potency):

Solutions of representative compounds of the present invention, at 3.3 mg/mL (active-form equivalents) were prepared at pH 4 (50 mM citrate) and pH 7 (50 mM phosphate). These solutions were stored in the dark and their visual clarity after 24 hours was determined, as is listed in Table 3, below. The recitation “yes” indicates the compound was soluble at 3.3 mg/mL at the designated pH after 24 hours; whereas the recitation “no” indicates the compound was not soluble at 3.3 mg/mL at the designated pH after 24 hours.

The aqueous solubility of Compounds #14, #15 and #16 were determined by a modified procedure. In these cases, the samples were targeted at a pH of 4 and 7 and were measured exclusively using 100 mM phosphate buffers. For both pH's, measured samples were initially prepared at ˜3 mg/ml and ˜10 mg/ml concentrations and for samples that dissolved rapidly, additional compound was added to the solution until saturated solutions were obtained. Concentrations reported as discrete values were determined by RP-HPLC and are also presented in Table 3, below.

The solubility of the parent compound (the compound of formula (M), also known as ((E)-7-(3-(2-amino-1-fluoroethylidene)piperidin-1-yl)-1-cyclopropyl-6-fluoro-8-methoxy-4-oxo-1,4-dihydroquinoline-3-carboxylic acid), was determined in a separate experiment, in which solutions containing 50 mM citrate buffer at various pH were added to the solid compound at various target concentrations. The samples were mixed overnight and the pH measured with a Beckman φ360 pH/Temp/mV Meter. The samples were then filtered using nylon centrifuge filters. The supernatant was diluted and the concentration was measured with HPLC. The solubility of the parent compound ((E)-7-(3-(2-amino-1-fluoroethylidene)piperidin-1-yl)-1-cyclopropyl-6-fluoro-8-methoxy-4-oxo-1,4-dihydroquinoline-3-carboxylic acid) was measured as 0.14 mg/mL at pH 4.11; and 0.04 mg/mL at pH 6.75.

TABLE 3
Aqueous Solubility

| ID No. | pH~4 | | pH~7 | |
|---|---|---|---|---|
| 1 | no | | No | |
| 2 | no | | No | |
| 3 | no | | No | |
| 4 | yes | | No | |
| 5 | no | | No | |
| 6 | no | | No | |
| 7 | yes | | Yes | |
| 8 | no | | No | |
| 9 | yes | | Yes | |
| 10 | no | | No | |
| 11 | no | | No | |
| 12 | NT^{a} | | NT^{a} | |
| 13 | no | | 1.8 | mg/mL @ pH 7.8 |
| 14 | 6.89 | mg/mL @ 18 h | >3 | mg/mL @ 18 h |
| 15 | >4.6 | mg/mL @ 18 h | >11.11 | mg/mL @ 18 h |
| 16 | >3.69 | mg/mL @ 18 h | >3.36 | mg/mL @ 18 h |

| | | |
|---|---|---|
| 17 | no | No |

^{a}Not Tested

### Example 19

### In Vitro Stability Studies

### Bioconversion of Pro-Drug Compounds to Parent Drug

Compounds #1, #4, #6, #15, and #16, as well as the parent drug [(E)-7-(3-(2-amino-1-fluoroethylidene)piperidin-1-yl)-1-cyclopropyl-6-fluoro-8-methoxy-4-oxo-1,4-dihydroquinoline-3-carboxylic acid] (the compound of formula (M)) were dissolved to 1 mg/mL, and diluted to 20 ng/mL in fresh, chilled (4° C.) human or mouse whole blood or plasma, or kidney and liver extract (prepared from the respective fresh mouse organs as described below). Compound concentrations were normalized to parent drug content by multiplying by a ratio of the relative molecular weights. Time 0 (minute) samples were prepared by immediately diluting with an equal volume of acetonitrile, vortexing, and transferring the samples to ice. For additional time points, samples were placed in an incubator (Tables 4 and 5), or in a heating block (Tables 6-12) at 37° C. for the indicated time, then immediately vortexed with an equal volume of acetonitrile and placed on ice. Samples were centrifuged and further diluted with an equal volume of water to a final concentration of 25% acetonitrile prior to analysis by HPLC; diluted samples were retained in the cooled (4° C.) HPLC sample injection chamber pending injection.

Representative compounds of the present invention and the parent compound were analyzed by reversed-phase HPLC using an Agilent 1100 system. Samples were injected onto a Zorbax SB-C18 column (3.5 μM, 4.6×150 mm) with a guard cartridge, and developed on a gradient of 15% aqueous acetonitrile in 0.1% trifluoroacetic acid to 85% aqueous acetonitrile in 0.1% trifluoroacetic acid over 10 minutes. The solvent flow rate was 1 mL/min, and the column temperature was 40° C. Compound and parent peak identification was by diode array detection, monitored at 300 nm Conversion of the compounds (prodrugs) to parent drug was estimated by peak areas (A₃₀₀ nm, mAU) of the respective agents.

Mouse whole blood and plasma were collected from Swiss Webster mice, and stored at 4° C. until use (within 48 hours). Tables 4, 5, 6 and 7, below lists results for Compounds #1, #6, #15 and #16, measured in mouse whole blood and plasma, as noted.

Tables 4 and 5, further present results with fresh whole mouse livers or kidneys. For these experiments, fresh whole mouse livers or kidneys were homogenized for approximately 30 seconds (Omni-Tip™ disposable homogenizer tip) in 1 mL sterile saline and immediately stored on ice. Crude extracts (non-centrifuged) were incubated with the test compound or parent (20 μg/mL) and processed as described above.

TABLE 4
Conversion of Compound #1 to Parent Drug
Fresh Mouse Plasma, Homogenized Liver or Homogenized Kidney Tissue

| | Peak Area (A₃₀₀, mAU) | | | | | |
|---|---|---|---|---|---|---|
| | Compound #1 | | | Compound (M) (Parent) | | |
| Time | Plasma | Liver | Kidney | Plasma | Liver | Kidney |
| 0 min | 298 | 235 | 222 | 0 | 24 | 27 |
| 5 min | 296 | 171 | 152 | 17 | 94 | 87 |
| 30 min | 286 | 51 | 41 | 23 | 220 | 203 |

TABLE 5
Conversion of the Compound #6 to Parent Drug
Fresh Mouse Plasma, Homogenized Liver or Homogenized Kidney Tissue

| | Peak Area (A₃₀₀, mAU) | | | | | |
|---|---|---|---|---|---|---|
| | Compound #6 | | | Compound (M) (Parent) | | |
| Time | Plasma | Liver | Kidney | Plasma | Liver | Kidney |
| 0 min | 363 | 254 | 219 | 24 | 48 | 73 |
| 5 min | 329 | 143 | 72 | 70 | 161 | 203 |
| 30 min | 281 | 34 | 11 | 107 | 283 | 286 |

TABLE 6
Conversion of Compound #15 to Parent Drug
Fresh Mouse Blood or Plasma

| | Peak Area (mAU, A₃₀₀ nm) | | | |
|---|---|---|---|---|
| Time | Compound #15 | | Compound (M) (Parent) | |
| (min) | Blood | Plasma | Blood | Plasma |
| 0 | 265 | 488 | 56 | 54 |
| 2.5 | 123 | 182 | 265 | 275 |
| 5 | 75 | 106 | 310 | 321 |
| 7.5 | 36 | 72 | 343 | 357 |
| 10 | 29 | 62 | 328 | 384 |
| 15 | 17 | 22 | 335 | 374 |

TABLE 7
Conversion of Compound #16 to Parent Drug, Fresh Mouse Blood or
Plasma, with Monitoring of a Peak of Unknown Identity

| | Peak Area (mAU, A₃₀₀ nm) | | | | | |
|---|---|---|---|---|---|---|
| | | | Compound (M) | | | |
| | Compound #16 | | (Parent) | | Unknown Peak | |
| Time (min) | Blood | Plasma | Blood | Plasma | Blood | Plasma |
| 0 | 332 | 406 | 12 | 35 | 148 | 70 |
| 2.5 | 174 | 285 | 138 | 36 | 142 | 161 |
| 5 | 131 | 220 | 195 | 40 | 103 | 219 |
| 7.5 | 99 | 178 | 257 | 46 | 77 | 262 |
| 10 | 71 | 149 | 281 | 41 | 55 | 268 |
| 15 | 34 | 98 | 330 | 42 | 27 | 298 |

For studies with Compounds #1, #4 and #6 (with results as listed in Tables 8, 9 and 10), fresh human whole blood and plasma samples were purchased from BioChemed Services, shipped in Styrofoam coolers maintained with frozen cold packs, and used within 48 hours of receipt. For studies with Compounds #15 and #16 (with results as listed in Tables 11 and 12), blood was drawn from volunteers and whole blood samples and isolated plasma prepared and immediately placed on ice. Whole blood and plasma samples were maintained at 4° C. and used within approximately 30 hours.

TABLE 8
Conversion of Compound #1 to Parent Drug
Fresh Human Blood or Plasma

| | Peak Area (mAU, A300 nm) | | | |
|---|---|---|---|---|
| Time | Compound #1 | | Compound (M) (Parent) | |
| (min) | Blood | Plasma | Blood | Plasma |
| 0.0 | 365 | 401 | 11 | 0 |
| 3.0 | 339 | | 46 | |
| 5.0 | | 386 | | 0 |
| 5.5 | 339 | | 60 | |
| 8.0 | 328 | | 89 | |
| 10.5 | 289 | | 93 | |
| 15.0 | | 369 | | 8.5 |
| 15.5 | 256 | | 129 | |
| 30.0 | | 371 | | 12 |

TABLE 9
Conversion of Compound #6 to Parent Drug
Fresh Human Blood or Plasma

| | Peak Area (mAU, A₃₀₀ nm) | | | |
|---|---|---|---|---|
| Time | Compound #6 | | Compound (M) (Parent) | |
| (min) | Blood | Plasma | Blood | Plasma |
| 0.0 | 369 | 393 | 12 | 0 |
| 3.5 | 290 | | 106 | |
| 5.0 | | 386 | | 0 |
| 6.0 | 188 | | 152 | |
| 8.5 | 159 | | 193 | |
| 11.0 | 134 | | 222 | |
| 15.0 | | 399 | | 6 |
| 16.0 | 95 | | 283 | |
| 30.0 | | 393 | | 9 |

TABLE 10
Conversion of Compound #4 to Parent Drug
Fresh Human Blood or Plasma

| | Peak Area (mAU, A₃₀₀ nm) | | | |
|---|---|---|---|---|
| Time | Compound #4 | | Compound (M) (Parent) | |
| (min) | Blood | Plasma | Blood | Plasma |
| 0.0 | 401 | 442 | 14 | 0 |
| 4.0 | 205 | | 220 | |
| 5.0 | | 383 | | 37 |
| 6.5 | 123 | | 310 | |
| 9.0 | 79 | | 320 | |
| 11.5 | 84 | | 348 | |
| 15.0 | | 339 | | 88 |
| 16.5 | 68 | | 352 | |
| 30.0 | | 268 | | 144 |

TABLE 11
Conversion of Compound #15 to Parent Drug
Fresh Human Blood or Plasma

| | Peak Area (mAU, A₃₀₀ nm) | | | |
|---|---|---|---|---|
| Time | Compound #15 | | Compound (M) (Parent) | |
| (min) | Blood | Plasma | Blood | Plasma |
| 0 | 242 | 330 | 38 | 44 |
| 2.5 | 59 | 75 | 233 | 290 |
| 5 | 23 | 26 | 275 | 315 |
| 7.5 | 10 | 14 | 290 | 348 |
| 10 | 8 | 10 | 299 | 342 |
| 15 | 6 | 11 | 319 | 362 |

TABLE 12
Conversion of the Compound 16 to Parent Drug, Fresh Human Blood or
Plasma, with Monitoring of a Peak of Unknown Identity

| | Peak Area (mAU, A₃₀₀ nm) | | | | | |
|---|---|---|---|---|---|---|
| | Compound | | Compound (M) | | | |
| | #16 | | (Parent) | | Unknown Peak | |
| Time (min) | Blood | Plasma | Blood | Plasma | Blood | Plasma |
| 0 | 370 | 351 | 0 | 0 | 47 | 0 |
| 2.5 | 336 | 350 | 0 | 0 | 75 | 0 |
| 5 | 332 | 355 | 2 | 0 | 80 | 0 |
| 7.5 | 342 | 366 | 2 | 0 | 72 | 0 |
| 10 | 374 | 372 | 4 | 0 | 61 | 0 |
| 15 | 359 | 371 | 8 | 0 | 60 | 0 |

The results listed in Tables 4-12 above indicate that Compounds #1, #4, and #6 are converted to the parent compound ([(E)-7-(3-(2-amino-1-fluoroethylidene)piperidin-1-yl)-1-cyclopropyl-6-fluoro-8-methoxy-4-oxo-1,4-dihydroquinoline-3-carboxylic acid]) in whole blood (human or mouse), liver/kidney extracts (mouse), and plasma (human or mouse). Compound #15 is converted to the parent in whole blood (human or mouse) and plasma (human or mouse). Compound #16 is converted to the parent in mouse whole blood via the intermediacy of a metabolite of unknown identity; whereas in mouse plasma, Compound #16 is converted to an unknown metabolite, but not substantially to the parent drug. Additionally, in human whole blood and plasma, Compound #16 was not measured to convert to the parent drug.

### Example 20

### In Vivo Efficacy in a Mouse Systemic Infection Model

Female Swiss Webster mice weighing between 20-25 g were infected intraperitoneally with approximately 5×10⁵ colony forming units (CFU) Staphylococcus aureus (OC8525), a methicillin-resistant strain (MRSA), in 7% mucin. One hour later, the animals were given the test compound intravenously or orally in a dose volume of 0.2 mL. Each animal test group consisted of eight animals. The test compounds were prepared immediately before dosing in 5% dextrose in water (D5W) and diluted further in D5W for i.v. and p.o. administration. The mice were observed over a three day period and ED₅₀ values were calculated from the resulting % survival curves (see Table 13, below). The measured ED₅₀ values were normalized to the content of (E)-7-(3-(2-amino-1-fluoroethylidene)piperidin-1-yl)-1-cyclopropyl-6-fluoro-8-methoxy-4-oxo-1,4-dihydroquinoline-3-carboxylic acid. For some compounds, in vivo efficacy was determined by testing a group of eight mice at a single dose level only. In these cases, the data presented in Table 13, below are reported as percent survival at the designated dose.

For comparison, the ED₅₀ values of the parent, (E)-7-(3-(2-amino-1-fluoroethylidene)piperidin-1-yl)-1-cyclopropyl-6-fluoro-8-methoxy-4-oxo-1,4-dihydroquinoline-3-carboxylic acid, were measured at 0.71 and 10.6 mg/kg/day when administered i.v. and p.o., respectively.

TABLE 13
in vivo Efficacy

| ID No. | ED₅₀ i.v. or % Survival | ED₅₀ p.o. |
|---|---|---|
| 1 | 1.3 | 9.9 |
| 2 | 2.6 | >14.9 |
| 3 | 1.4 | >14.8 |
| 4 | 0.7 | 19.8 |
| 5 | >3.9 | >15.7 |
| 6 | 0.6 | >17.6 |
| 7 | 0% at 2.1 mg/kg/day | NT^{a} |
| 8 | NT^{a} | NT^{a} |
| 9 | 25% at 1.9 mg/kg/day | NT^{a} |
| 10 | 25% at 2.4 mg/kg/day | NT^{a} |
| 11 | 1.2 | 6.7 |
| 12 | NT^{a} | NT^{a} |
| 13 | 12.5% at 3.8 mg/kg/day | NT^{a} |
| 14 | 87.5% at 3.9 mg/kg/day | NT^{a} |
| | 12.5% at 2.0 mg/kg/day | |
| 15 | 1.6 | >13.0 |
| 16 | 1.6 | >13.9 |
| 17 | 25% at 0.78 mg/kg/day | NT^{a} |
| | 0% at 0.39 mg/kg/day | |

^{a}Not Tested

### Example 21

### Rat Pharmacokinetics

Male Sprague-Dawley rats (n=4) were fasted overnight, then dosed intravenously (IV) with a 1 mg/mL solution of a compound of the present invention (prodrug) or parent (compound of formula (M)) in 20% hydroxypropyl-β-cyclodextrin at 2 mg/kg. Blood samples were collected up to 24 hours post dose into tubes containing an anticoagulant. Blood samples were centrifuged for cell removal, and 100 μL plasma was transferred to a clean vial, placed on dry ice, and stored in a −70° C. freezer prior to analysis.

Plasma samples were prepared as follows. Two volumes of acetonitrile containing formic acid and internal standard were added to one volume of plasma to precipitate proteins. Samples were centrifuged (3000 g for 5 min) and supernatant removed for analysis by LC-MS-MS. Calibration standards were prepared by adding appropriate volumes of stock solution directly into plasma and treated identically to collected plasma samples. LC-MS-MS analysis was performed utilizing multiple reaction monitoring for detection of characteristic ions for the test compounds (pro-drugs), parent (compound of formula (M)) and internal standard.

Plasma concentrations were measured as described above to determine a concentration vs time profile. The area under the plasma concentration vs time curve (AUC) was calculated using the linear trapezoidal method. Fitting of the data to obtain pharmacokinetic parameters was carried out using non-compartmental analysis, with results as listed in Table 14, below. The term “NA” means the data was not available.

TABLE 14
Mean plasma pharmacokinetic parameters
In male rats after administration of 2 mg/kg i.v.

| Compound | | Compound #15 | | Compound #16 | |
|---|---|---|---|---|---|
| Analyte | Parent^{a} | 15 | parent | 16 | parent |
| Cₘₐₓ or Cₒ [ng/ml] | 805 | 30 | 319 | 829 | 193 |
| AUC [ng · h/ml] | 718 | 99 | 310 | 83.5 | 262 |
| T_{1/2} [h] | 1.50 | NA | 1.04 | NA | 1.30 |
| CL [mL/min/kg] | 47.1 | NA | | NA | |
| Vₛₛ [L/kg] | 6.08 | 151 | | 3.75 | |
| Bioavailability of | 100 | 66.4 | | 52.8 | |
| active [%] | | | | | |

^{a}(E)-7-(3-(2-amino-1-fluoroethylidene)piperidin-1-yl)-1-cyclopropyl-6-fluoro-8-methoxy-4-oxo-1,4-dihydroquinoline-3-carboxylic acid

The results listed in Table 14 above, indicate that Compounds #15 and #16 are rapidly converted to the parent drug upon intravenous administration. The exposure of the active component was measured at 66.4% for Compound #15 and 52.8% for Compound #16, when compared to normalized equivalent doses of the parent.

### Example 22

### Prophetic Example, Oral Solid Dosage Form

As a specific embodiment of an oral composition, 100 mg of the compound prepared as in Example 6 is formulated with sufficient finely divided lactose to provide a total amount of 580 to 590 mg to fill a size O hard gel capsule.

### Example 23

### Prophetic Example, Parenteral Dosage Form

As a specific embodiment of a parenteral dosage composition, 750 mg of Compound #15, prepared as in Example 6 is formulated in 150 mL of 5% dextrose aqueous solution at pH 4.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed:

1. A compound of formula (I) 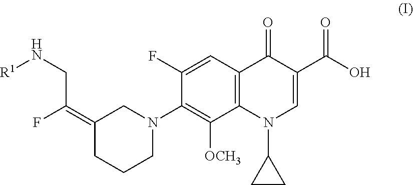
wherein
R¹ is selected from the group consisting of 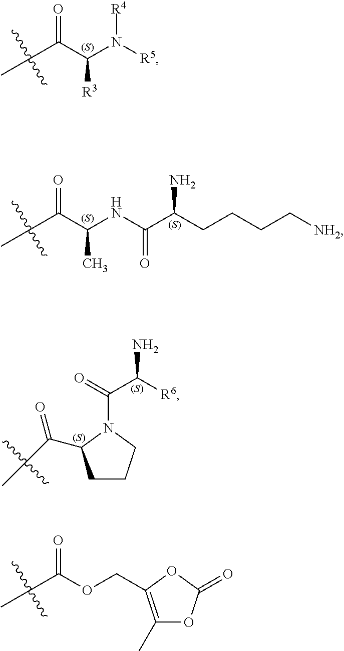
and —P(O)(OR⁷)₂;
R³ is selected from the group consisting of hydrogen, lower alkyl, benzyl, —CH₂CO₂H, —(CH₂)₄NH₂ and —CH₂N(CH₃)₂;
R⁴ is selected from the group consisting of hydrogen and lower alkyl;
R⁵ is selected from the group consisting of hydrogen, 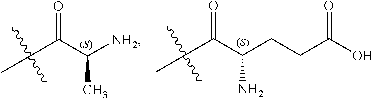
and —C(O)—(CH₂)₂—C(O)-mPEG(2000);
R⁶ is selected from the group consisting of lower alkyl and —(CH₂)₄—NH₂;
each R⁷ is independently selected from lower alkyl;
and pharmaceutically acceptable salts thereof.

2. A compound as in claim 1, wherein
R¹ is selected from the group consisting of 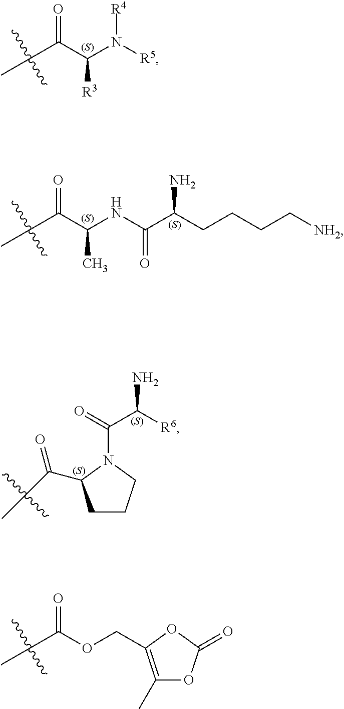
and —P(O)(OR⁷)₂;
R³ is selected from the group consisting of hydrogen, lower alkyl, benzyl, —CH₂CO₂H, —(CH₂)₄NH₂ and —CH₂N(CH₃)₂;
R⁴ is selected from the group consisting of hydrogen and lower alkyl;
R⁵ is selected from the group consisting of hydrogen. 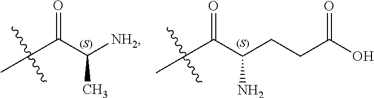
and —C(O)—(CH₂)₂—C(O)-mPEG(2000);
R⁶ is selected from the group consisting of lower alkyl and —(CH₂)₄—NH₂;
each R⁷ is selected from the group consisting of lower alkyl;
or a pharmaceutically acceptable salt thereof.

3. A compound as in claim 2, wherein
R¹ is selected from the group consisting of 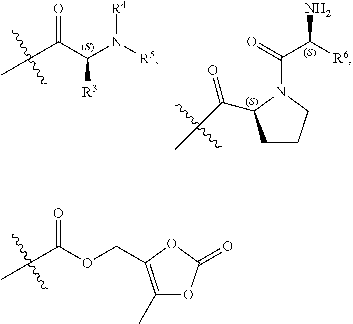
and —P(O)(OR⁷)₂;
R³ is selected from the group consisting of hydrogen, methyl, isopropyl, isobutyl, benzyl, —CH₂CO₂H, —(CH₂)₄NH₂ and —CH₂N(CH₃)₂;
R⁴ is selected from the group consisting of hydrogen and methyl;
R⁵ is selected from the group consisting of hydrogen, 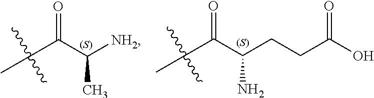
and —C(O)—(CH₂)₂—C(O)-mPEG(2000);
R⁶ is selected from the group consisting of methyl, isopropyl and —(CH₂)₄—NH₂;
each R⁷ is selected from the group consisting of lower alkyl;
or a pharmaceutically acceptable salt thereof.

4. A compound as in claim 3, wherein
R¹ is selected from the group consisting of 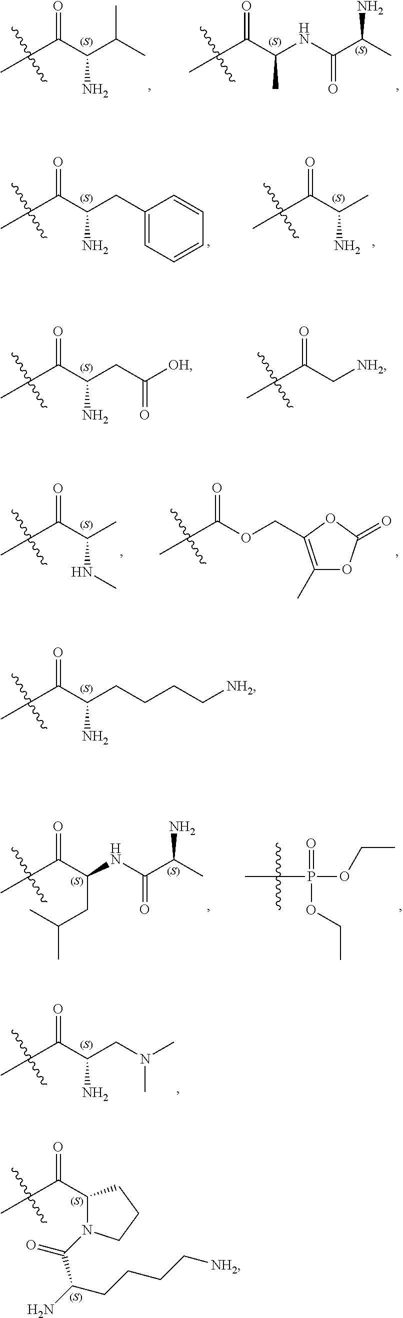 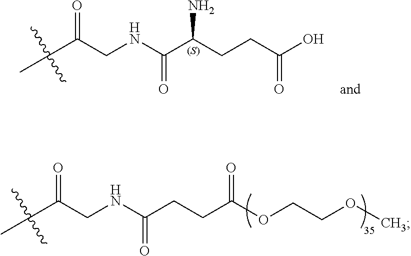
or a pharmaceutically acceptable salt thereof.

5. A compound as in claim 4, wherein
R¹ is selected from the group consisting of 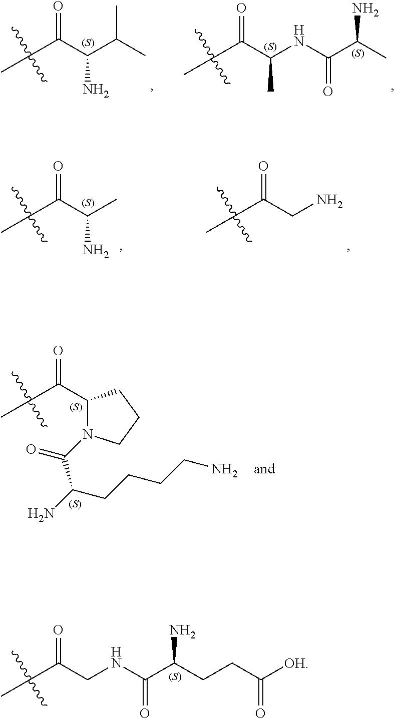
or a pharmaceutically acceptable salt thereof.

6. A compound as in claim 4, selected from the group consisting of
1-Cyclopropyl-7-[3-(2-{[(2S)-1-((2S)-2,6-diamino-hexanoyl)-pyrrolidine-2-carbonyl]-amino}-1-fluoro-ethylidene)-piperidin-1-yl]-6-fluoro-8-methoxy-4-oxo-1,4-dihydro-quinoline-3-carboxylic acid;
7-(3-{2-[2-((2S)-2-Amino-4-carboxy-butyrylamino)-acetylamino]-1-fluoro-ethylidene}-piperidin-1-yl)-1-cyclopropyl-6-fluoro-8-methoxy-4-oxo-1,4-dihydro-quinoline-3-carboxylic acid; and
and pharmaceutically acceptable salts thereof.

7. A compound as in claim 6, selected from the group consisting of
1-Cyclopropyl-7-[3-(2-{[(2S)-1-((2S)-2,6-diamino-hexanoyl)-pyrrolidine-2-carbonyl]-amino}-1-fluoro-ethylidene)-piperidin-1-yl]-6-fluoro-8-methoxy-4-oxo-1,4-dihydro-quinoline-3-carboxylic acid dihydrochloride; and
7-(3-{2-[2-((2S)-2-Amino-4-carboxy-butyrylamino)-acetylamino]-1-fluoro-ethylidene}-piperidin-1-yl)-1-cyclopropyl-6-fluoro-8-methoxy-4-oxo-1,4-dihydro-quinoline-3-carboxylic acid hydrochloride.

8. A compound selected from the group consisting of
1-Cyclopropyl-7-[3-(2-{[(2S)-1-((2S)-2,6-diamino-hexanoyl)-pyrrolidine-2-carbonyl]-amino}-1-fluoro-ethylidene)-piperidin-1-yl]-6-fluoro-8-methoxy-4-oxo-1,4-dihydro-quinoline-3-carboxylic acid;
and pharmaceutically acceptable salts thereof.

9. A pharmaceutical composition comprising a pharmaceutically acceptable carrier and a compound of claim 1.

10. A pharmaceutical composition made by mixing a compound of claim 1 and a pharmaceutically acceptable carrier.

11. A process for making a pharmaceutical composition comprising mixing a compound of claim 1 and a pharmaceutically acceptable carrier.

12. A method of treating a subject having a condition caused by or contributed to by bacterial infection, comprising administering to a subject in need thereof a therapeutically effective amount of the compound as in claim 1.

13. A method of preventing a subject from suffering from a condition caused by or contributed to by bacterial infection, comprising administering to a subject in need thereof a prophylactically effective dose of a compound as in claim 1.

14. The use of a compound as in claim 1 for the preparation of a medicament for treating or preventing a condition caused by or contributed to by bacterial infection, in a subject in need thereof.

15. A compound of formula (II) 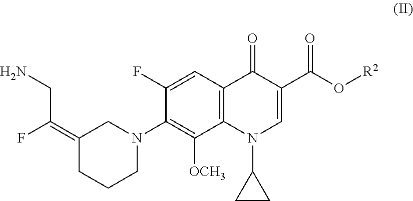
wherein
R² is lower alkyl;
or a pharmaceutically acceptable salts thereof.

16. A compound as in claim 15, wherein R² is selected from the group consisting of methyl and isopropyl.

17. A pharmaceutical composition comprising a pharmaceutically acceptable carrier and a compound of claim 15.

18. A pharmaceutical composition made by mixing a compound of claim 15 and a pharmaceutically acceptable carrier.

19. A process for making a pharmaceutical composition comprising mixing a compound of claim 15 and a pharmaceutically acceptable carrier.

20. A method of treating a subject having a condition caused by or contributed to by bacterial infection, comprising administering to a subject in need thereof a therapeutically effective amount of the compound as in claim 15.

21. A method of preventing a subject from suffering from a condition caused by or contributed to by bacterial infection, comprising administering to a subject in need thereof a prophylactically effective dose of a compound as in claim 15.

22. The use of a compound as in claim 15 for the preparation of a medicament for treating or preventing a condition caused by or contributed to by bacterial infection, in a subject in need thereof.
